SEQUENCE LISTING (6 sequences)

SEQ ID NO: 1 - DNA, 20 bp, Artificial Sequence
  Synthesized DNA
tgaacagtggtagcgacagc

SEQ ID NO: 2 - DNA, 19 bp, Artificial Sequence
  Synthesized DNA
ggagctatctcggtgcctg

SEQ ID NO: 3 - DNA, 24 bp, Artificial Sequence
  Synthesized DNA
atccccaagccaaagctacaggga

SEQ ID NO: 4 - DNA, 21 bp, Artificial Sequence
  Synthesized DNA
cagcagatgtggatcagcaag

SEQ ID NO: 5 - DNA, 18 bp, Artificial Sequence
  Synthesized DNA
gcatttgcggtggacgat

SEQ ID NO: 6 - DNA, 23 bp, Artificial Sequence
  Synthesized DNA
aggagtatgacgagtccggcccc

(19) United States
(12) Patent Application Publication
(10) Pub. No.: US 2013/0274127 A1
(43) Pub. Date: Oct. 17, 2013

(54) GENE EXPRESSION MARKERS FOR PREDICTION OF RESPONSE TO PHOSPHOINOSITIDE 3-KINASE INHIBITORS

(71) Applicant: GENOMIC HEALTH, INC., Redwood City, CA (US)

(72) Inventors: Joffre B. Baker, Montara, CA (US); Robert J. Pelham, Belmont, CA (US); Kevin A. Kwei, Mountain View, CA (US)

(73) Assignee: GENOMIC HEALTH, INC., Redwood City, CA (US)

(21) Appl. No.: 13/804,351

(22) Filed: Mar. 14, 2013

Related U.S. Application Data
(60) Provisional application No. 61/625,262, filed on Apr. 17, 2012.

(51) Int. Cl. C12Q 1/68

(52) U.S. Cl. CPC C12Q 1/6886
USPC 506/9; 435/6.12; 435/375; 435/7.9

(57) ABSTRACT

The present invention provides methods for predicting a likelihood that a tumor cell will be sensitive or resistant to a phosphoinositide 3-kinase (PI3K) inhibitor. The methods generally involve determining an expression level of a gene product that correlates with sensitivity or resistance to a PI3K inhibitor. The present invention also provides methods for increasing sensitivity of a tumor cell to a PI3K inhibitor by contacting the tumor cell with an activator or inhibitor of a gene product that correlates with sensitivity or resistance to a PI3K inhibitor.

### RELATED APPLICATION

This application claims the benefit of priority of U.S. Provisional Application No. 61/625,262, filed Apr. 17, 2012, and which is hereby incorporated by reference in its entirety.

### FIELD OF THE INVENTION

The present invention relates to genes, the expression levels of which are useful for predicting response of tumor cells and cancer patients to a phosphoinositide 3-kinase (PI3K) inhibitor.

### BACKGROUND

The PI3K (phosphoinositide 3-kinase) signaling cascade is one of the most frequently de-regulated pathways in human cancer (Vivanco et al., Nat. Rev. Cancer, 2:489-501, 2002), resulting in aberrant cell proliferation and migration. Activation by upstream receptor tyrosine kinases results in the PI3K-mediated conversion of phosphatidylinositol diphosphate (PIP2) into the secondary messenger phosphatidylinositol triphosphate (PIP3) (Liu et al., Nat. Rev. Drug Discov. 8:627-644, 2009; Folkes et al., J. Med. Chem. 51:5522-5532, 2008; Raynaud et al., Cancer Res. 67:5840-5850, 2007). PIP3 subsequently recruits 3-phosphoinositide dependent protein kinase 1 (Pdpk1) and the serine-threonine protein kinase, Akt, to the plasma membrane, resulting in the phosphorylation and activation of Akt. It is Akt which then mediates a cascade of phosphorylation events leading to the activation of downstream pathways resulting in pro-tumor survival signaling and poor prognosis in cancer patients (Vivanco et al., Nat. Rev. Cancer, 2:489-501, 2002; Engelman et al., Nat. Rev. Cancer 9:550-562, 2009). Perturbations in multiple components of the PI3K-Akt signaling axis have been observed in numerous tumor types, most notable of which are mutations in PIK3CA, the gene encoding the p110α catalytic subunit of the class I PI3K. Additional mutations include amplifications or activating mutations in PI3KCB, PDPK1, and AKT1, and loss of heterozygosity and mutational inactivation of PTEN, a negative regulator of PI3K-signaling (Vivanco et al., Nat. Rev. Cancer, 2:489-501, 2002; Engelman et al., Nat. Rev. Cancer 9:550-562, 2009; Liu et al., Nat. Rev. Drug Discov. 8:627-644, 2009).

Given the frequent activation of PI3K, and its downstream effectors, there is interest in developing selective inhibitors against the PI3K pathway as a chemotherapeutic strategy in cancer, and a number of these compounds are undergoing clinical development (Engelman et al., Nat. Rev. Cancer 9:550-562, 2009). One of these PI3K inhibitors is GDC-0941, an orally bio-available small molecule inhibitor that that selectively targets all class I PI3K isoforms (Folkes et al., J. Med. Chem. 51:5522-5532, 2008). Several pre-clinical studies of GDC-0941, including one study using a panel of 54 breast cancer cell lines, have demonstrated its potent anti-tumor activities in cell lines that harbor activating mutations in PI3KCA, and the use of P13KCA as a positive predictor of sensitivity to GDC-0941 and other inhibitors of PI3K (Raynaud et al., Cancer Res. 67:5840-5850, 2007; O'Brien et al., Clin. Cancer Res. 16:3570-3683, 2010; Dan et al., Cancer Res. 70:4982-4994, 2010; Wallin et al., Mol. Cancer Ther. 10:2426-2436, 2011). Interestingly, these studies have also shown that mutation status alone does not predict sensitivity, as a number of cell lines lacking an activated PI3K pathway are still sensitive to GDC-0941. A better understanding of the biology that underlies sensitive versus resistant phenotypes has the potential to impact the clinical utility of GDC-0941 and other drugs targeting the PI3K-Akt signaling axis by facilitating the selection of those patients most likely to derive benefit.

### SUMMARY

The present invention provides response indicator genes for PI3K inhibitors. These genes are provided in Tables 2-4. In an embodiment of the invention, increased expression level of one or more response indicator genes selected from Table 2 is positively correlated with a likelihood that a tumor cell will be sensitive to a PI3K inhibitor. In another embodiment of the invention, increased expression level of one or more response indicator genes selected from Table 3 is positively correlated with a likelihood that a tumor cell will be resistant to a PI3K inhibitor.

In a specific embodiment of the invention, increased expression level of one or more genes selected from VAV3, DGKE, MBD1, SERP1, TXNL1, NLGN4X, C1ORF91, SLC25A1, ZBTB40, FAM51A1, LOC116349, KIAA1468, PIGN, PKD1L1, SELT, CISH, MGC50559, NPY5R, PEX10, and C6ORF35 is positively correlated with a likelihood that a tumor cell will be sensitive to a PI3K inhibitor. In another embodiment, increased expression level of one or more genes selected from TRIM50C, GALR2, INSL3, LOC389633, GTPBP10, PGRMC1, DNASE1L3, CACNG2, FAM90A1, OGT, FKBP6, GDAP1L1, CHRNB1, NLGN3, ZNF259, DDN, NXF3, MGC35366, TANK, and LOC116123 is positively correlated with a likelihood that a tumor cell will be resistant to a PI3K inhibitor.

The present invention further provides methods and compositions for predicting the likelihood that a tumor cell will be sensitive or resistant to a PI3K inhibitor based on the expression level of one or more response indicator genes in the tumor cell. Specifically, the method comprises assaying or measuring an expression level of one or more response indicator gene products. The response indicator gene is selected from any one of the genes listed in Tables 2-4. In an embodiment of the invention, the expression level of the response indicator gene is normalized. The expression level or the normalized expression level is used to predict the likelihood that a tumor cell will be sensitive or resistant to a PI3K inhibitor, wherein increased expression level or increased normalized expression level of one or more response indicator genes selected from Table 2 is positively correlated with a likelihood that the tumor cell will be sensitive to a PI3K inhibitor, and increased expression level or increased normalized expression level of one or more response indicator genes selected from Table 3 is positively correlated with a likelihood that the tumor cell will resistant to a PI3K inhibitor. In a specific embodiment of the invention, an increased expression level of one or more genes selected from VAV3, DGKE, MBD1, SERP1, TXNL1, NLGN4X, C1ORF91, SLC25A1, ZBTB40, FAM51A1, LOC116349, KIAA1468, PIGN, PKD1L1, SELT, CISH, MGC50559, NPY5R, PEX10, and C6ORF35 is positively correlated with a likelihood that a tumor cell will be sensitive to a PI3K inhibitor. In another embodiment, an increased expression level of one or more genes selected from TRIM50C, GALR2, INSL3, LOC389633, GTPBP10, PGRMC1, DNASE1L3, CACNG2, FAM90A1, OGT, FKBP6, GDAP1L1, CHRNB1, NLGN3, ZNF259, DDN, NXF3, MGC35366, TANK, and LOC116123 is positively correlated with a likelihood that a tumor cell will be resistant to a PI3K inhibitor. In a further embodiment of the invention, a report is generated based on the predicted likelihood of response.

The methods of the present invention contemplate determining the expression level of at least one response indicator gene or its gene product. For all aspects of the present invention, the methods may further include determining the expression levels of at least two response indicator genes, or their expression products. It is further contemplated that the methods of the present disclosure may further include determining the expression levels of at least three response indicator genes, or their expression products. It is contemplated that the methods of the present disclosure may further include determining the expression levels of at least four response indicator genes, or their expression products. It is contemplated that the methods of the present disclosure may further include determining the expression levels of at least five response indicator genes, or their expression products. It is contemplated that the methods of the present disclosure may further include determining the expression levels of at least six response indicator genes, or their expression products. It is contemplated that the methods of the present disclosure may further include determining the expression levels of at least seven response indicator genes, or their expression products. It is contemplated that the methods of the present disclosure may further include determining the expression levels of at least eight response indicator genes, or their expression products. It is contemplated that the methods of the present disclosure may further include determining the expression levels of at least nine response indicator genes, or their expression products. The methods may involve determination of the expression levels of at least ten (10) or at least fifteen (15) of the response indicator genes, or their expression products.

The expression level, or normalized expression level, of the response indicator gene, or its expression product, is used to predict the likelihood that a tumor cell will be sensitive or resistant to a PI3K inhibitor. In an embodiment of the invention, a likelihood score (e.g., a score predicting a likelihood that a tumor cell will be resistant to a PI3K inhibitor) can be calculated based on the expression level or normalized expression level. A score may be calculated using weighted values based on the expression level or normalized expression level of a response indicator gene and its contribution to response to a PI3K inhibitor.

In an embodiment of the invention, the expression product of the response indicator gene to be assayed or measured is an RNA transcript. In one aspect, the RNA transcripts are fragmented. In another embodiment, the expression product is a polypeptide. Determining the expression level of one or more response indicator gene products may be accomplished by, for example, a method of gene expression profiling. The method of gene expression profiling may be, for example, a sequencing or PCR-based method. The expression level of said genes can be determined, for example, by RT-PCR (reverse transcriptase PCR), quantitative RT-PCR (qRT-PCR), or other PCR-based methods, immunohistochemistry, proteomics techniques, an array-based method, or any other methods known in the art or their combination.

The tumor cell may be, for example, a tumor cell or tumor cells obtained from a tumor tissue sample. In an embodiment of the invention, the tumor cell is obtained from fixed and paraffin-embedded, fresh, or frozen tissue. For example, the tissue may be from a biopsy (fine needle, core or other types of biopsy) or obtained by fine needle aspiration, or by obtaining body fluid containing a tumor cell, e.g. urine, blood, etc. In an embodiment of the invention, the tumor cell is selected from a breast, colon, non-small cell lung, ovarian, prostate, and melanoma tumor cell.

The present invention further provides a method of increasing sensitivity of a tumor cell to a PI3K inhibitor. In an embodiment of the invention, the method comprises contacting the tumor cell with an inhibitor of one or more genes selected from Table 3, and contacting the tumor cell with a PI3K inhibitor. In another embodiment, the method comprises contacting the tumor cell with an activator of one or more genes selected from Table 2, and contacting the tumor cell with a PI3K inhibitor.

In any of the embodiments of the present invention, the PI3K inhibitor may be GDC-0941.

### BRIEF DESCRIPTION OF THE DRAWINGS

FIGS. 1A-1D show the sensitivity of the NCI-60 tumor cell line collection to GDC-0941. A. GI₅₀ (in μM) of GDC-0941 for 60 cell lines are ordered from lowest to highest, with cell lines containing the PIK3CA mutation are shaded. B. z-scores of cell line sensitivity are ordered from lowest to highest. z-scores of 0.8 and −0.8 are indicated by the dashed light line. C. GI₅₀ (in μM) of GDC-0941 for 16 sensitive and resistant cell lines of epithelia origin are ordered from lowest to highest. D. z-scores of cell line sensitivity for 16 sensitive and resistant cell lines of epithelia origin are ordered from lowest to highest. z-scores of 0.8 and −0.8 are indicated by the dashed light line.

FIG. 2 shows the sensitivity of the NCI-60 tumor cell line collection to GDC-0941. GI₅₀ (in μM) of GDC-0941 for 60 cell lines are organized by tumor of origin, as indicated below cell line names. Mean GI₅₀ (0.66 μM) for all cell lines is indicated by a dashed line.

FIGS. 3A-3C show the correlation between the sensitivity of the NCI-60 tumor cell line collection to GDC-0941 and mutational status of PI3KCA, PTEN, and tumor type. A. Correlation between GI₅₀ (μM) to GDC-0941 and mutational status of PI3KCA or PTEN, in all 60 tumor cell lines in the NCI-60 collection. B. Correlation between GI₅₀ (μM) to GDC-0941 and mutational status of PI3KCA or PTEN, in 16 GDC-0941-resistant and -sensitive tumor cell lines (IGROV1, T-47D, UACC-257, MCF-7, BT-549, HOP-92, HS578T, SK-OV-3, M14, SK-MEL-5, HCC-2998, NCI-H226, MDA-MB-231, NCI-H23, NCI/ADR-RES, OVCAR-4) analyzed in detail in this study. C. Correlation between GI₅₀ (μM) to GDC-0941 and tumor type of all 60 tumor cell lines in the NCI-60 collection.

FIGS. 4A-4C show the gene expression signature predictive of tumor cell response to GDC-0941. A. Gene expression signature identifies tumor cells that are sensitive (“S”) or resistant (“R”) to GDC-0941. Unsupervised hierarchical clustering was performed for 50 genes most differentially expressed between 16 GDC-0941 sensitive (n=8) and resistant (n=8) tumor cell lines (as determined by the Comparative Marker Selection suite in GenePattern with a P value <0.01). Cell lines are shown on the horizontal axis and genes are shown on the vertical axis. Color bar indicates relative levels (log₂) of gene expression, following median centering. B and C. Correlation between tumor cell sensitivity to GDC-0941 and mRNA expression levels of OGT and DDN, respectively. Data in scatter plots (left panels) represent mean log₂ mRNA expression of OGT or DDN (y-axis) versus GI₅₀ (μM) of GDC-041 (x-axis) for each of 16 cell lines (n=3 for each cell line tested). Correlation between log₂ mRNA expression and GI₅₀ as estimated by Spearman's rank correlation (rₛ) is indicated. Data in box plots (right panels) represents mean±SD log₂ level of mRNA expression of OGT or DDN in all sensitive cell lines (n=8) versus all resistant cell lines (n=8);*, P<0.05;**, P<0.01.

FIGS. 5A-5C show a Gene Set Enrichment Analysis (GSEA), which identified biologically-coherent pathways differentially expressed between GDC-0941-resistant and-sensitive tumor cell lines. A. Table of KEGG pathways upregulated in GDC-0941-resistant tumor cell lines, with number of genes (“SIZE”), enrichment score (“ES”), normalized enrichment score (“NES”), nominal p-value (“NOM p-val”), and q-value (“FDR q-val”) listed. B. Enrichment plot indicating the enrichment score for the KEGG proteasome gene set. C. Heatmap showing relative expression of genes in KEGG proteasome gene set amongst sixteen GDC-0941-resistant and—sensitive tumor cell lines.

FIGS. 6A-6E show the relationship between de novo phosphorylation status of multiple signaling pathways and tumor cell sensitivity to GDC-0941. A. Relative activities of multiple signaling pathways in tumor cell lines with differential sensitivities to GDC-0941. The heat map indicates the relative phosphorylation levels of Erk½ (T202/Y204), Mek½ (S217/S221), p38 MAPK (T180/Y182), Stat3 (Tyr705), Erbb2 (Y1221/Y1222), Erbb3 (panY), Akt1 (Ser473), Akt1 (T308), mTOR (S2448), 4E-BP1 (T37/T46), p70 S6 Kinase (T389), S6 (S235/S236), IRS1 (S307), IRS2 (panY), p53 (S15), Bad (S112), cleaved caspase-3, cleaved PARP, and Chk2 (T68) as assessed by quantitative phosphorylation analysis using a sandwich ELISA with epitope-specific antibodies. Cell lines are ordered (left to right) in increasing order of resistance, with sensitivity of each cell line indicated below cell line label as sensitive “S” or resistant “R”. Color bar indicates relative levels (z-score) of phosphorylation. B-E. Correlation between tumor cell sensitivity to GDC-0941 and phosphorylation status of Akt1 (Ser473), Akt1 (T308), 4E-BP1 (T37/T46), and cleaved PARP, respectively. Data in scatter plots (left panels) represent z-score of phosphorylation status of (y-axis) versus GI₅₀ (μM) of GDC-041 (x-axis) for each of 16 cell lines (n=3 for each cell line tested). Correlation between z-score and GI₅₀ as estimated by Spearman's rank correlation (rₛ) is indicated. Data in box plots (right panels) represents mean±SEM relative level of phosphorylation for indicated protein, for all sensitive cell lines (n=8) versus all resistant cell lines (n=8);*, P<0.05;***, P<0.001.

FIGS. 7A-7F show that loss of OGT expression increases sensitivity of the MDA-MB-231 tumor cell line to GDC-0941 and alters the phosphorylation state of mTOR, Chk2, and p38 MAPK. A. MDA-MB-231 cells transfected with OGT-targeting siRNA or non-silencing siRNA, in the presence or absence of 1 μM GDC-0941. Cell viability was assayed at 0, 24, 48, and 72 hours post-transfection and treatment with GDC-0941. B. Immunoblot analysis of whole cell lysates from MDA-MB-231 cells using anti-OGT or anti-OGlcNAc antibodies. Cells were treated with treated with 1 μM GDC-0941 or DMSO control for 24 hours, as indicated. C. Immunoblot analysis of whole cell lysates from MDA-MB-231 cells with indicated antibodies. Cell treatments are as indicated. D-E. Quantitative phosphorylation analysis of MDA-MB-231 cells transfected with OGT-targeting siRNA or non-silencing siRNA, in the presence or absence of 1 μM GDC-0941 for 24 hours. Cells were treated with treated with 1 μM GDC-0941 or DMSO control for 24 hours, as indicated, with phospho-specific epitopes indicated above each graph. All of the above data represent mean±SEM (n=3);*, P<0.05;**, P<0.01;***, P<0.001.

FIGS. 8A-8F show that loss of OGT expression increases sensitivity of the OVCAR-4 tumor cell line to GDC-0941 and alters cleavage status of caspase-3 as well as the phosphorylation state of Chk2 and p38 MAPK. A. OVCAR-4 cells transfected with OGT-targeting siRNA or non-silencing siRNA, in the presence or absence of 4 μM GDC-0941. Cell viability was assayed at 0, 24, 48, and 72 hours post-transfection and treatment with GDC-0941. B. Immunoblot analysis of whole cell lysates from OVCAR-4 cells using anti-OGT and anti-OGlcNAc antibodies. Cells were treated with treated with 4 μM GDC-0941 or DMSO control for 24 hours, as indicated. C. Immunoblot analysis of whole cell lysates from OVCAR-4 cells with indicated antibodies. Cell treatments are as indicated. D-E. Quantitative phosphorylation analysis of OVCAR-4 cells transfected with OGT-targeting siRNA or non-silencing siRNA, in the presence or absence of 4 μM GDC-0941 for 24 hours. Cells were treated with treated with 4 μM GDC-0941 or DMSO control for 24 hours, as indicated, with phospho-specific epitopes indicated above each graph. All of the above data represent mean±SEM (n=3);*, P<0.05;**, P<0.01;***, P<0.001.

FIGS. 9A-9D show the quantitative analysis of O-GlcNAc levels in OGT siRNA-treated cells, as determined by Western blotting. A. Pixel density of total O-GlcNAc levels for MDA-MB-231 tumor cells transfected with OGT-targeting siRNA or non-silencing siRNA, in the presence or absence of 1 μM GDC-0941 for 24 hours. Each treatment condition is indicated below the x-axis. B. % change in pixel density of total O-GlcNAc levels for MDA-MB-231 tumor cells transfected with OGT-targeting siRNA or non-silencing siRNA, in the presence or absence of 1 μM GDC-0941 for 24 hours, relative to NS siRNA-treated cells. C. Pixel density of total O-GlcNAc levels for OVCAR-4 tumor cells transfected with OGT-targeting siRNA or non-silencing siRNA, in the presence or absence of 4 μM GDC-0941 for 24 hours. Each treatment condition is indicated below the x-axis. D. % change in pixel density of total O-GlcNAc levels for OVCAR-4 tumor cells transfected with OGT-targeting siRNA or non-silencing siRNA, in the presence or absence of 4 μM GDC-0941 for 24 hours, relative to non-silencing siRNA-treated cells. Data in A and C represent mean±SD (n=2).

FIG. 10 shows the quantitative phosphorylation analysis of MDA-MB-231 cells transfected with OGT-targeting siRNA or non-silencing siRNA, in the presence or absence of 1 μM GDC-0941 for 24 hours. Cells were treated with 1 μM GDC-0941 or DMSO control for 24 hours, as indicated, with phospho-specific epitopes indicated above each graph. All of the above data represent mean±SEM (n=3);*, P<0.05;**, P<0.01;***, P<0.001.

FIG. 11 shows the quantitative phosphorylation analysis of OVCAR-4 cells transfected with OGT-targeting siRNA or non-silencing siRNA, in the presence or absence of 4 μM GDC-0941 for 24 hours. Cells were treated with 4 μM GDC-0941 or DMSO control for 24 hours, as indicated, with phospho-specific epitopes indicated above each graph. All of the above data represent mean±SEM (n=3);*, P<0.05;**, P<0.01;***, P<0.001.

FIGS. 12A-12B show loss of OGT expression increased sensitivity of the MDA-MB-231 tumor cell line but not the OVCAR-4 tumor cell line to the dual PI3K/mTOR inhibitor NVP-BEZ235. A. MDA-MB-231 cells transfected with OGT-targeting siRNA or non-silencing siRNA, in the presence or absence of 10 nM NVP-BEZ235. Cell viability was assayed at 0, 24, 48, and 72 hours post-transfection and treatment with NVP-BEZ235. B. OVCAR-4 cells transfected with OGT-targeting siRNA or non-silencing siRNA, in the presence or absence of 10 nM NVP-BEZ235. Cell viability was assayed at 0, 24, 48, and 72 hours post-transfection and treatment with NVP-BEZ235. All above data represent mean±SEM (n=3);*, P<0.05;**, P<0.01;***, P<0.001.

FIGS. 13A-13J show loss of DDN expression increased sensitivity of the MCF-7 and MDA-MB-231 tumor cell lines to GDC-0941 and altered the phosphorylation state of p38 MAPK. A. MCF-7 cells transfected with DDN-targeting siRNA or non-silencing siRNA, in the presence or absence of 300 nM GDC-0941. Cell viability was assayed at 0, 24, 48, and 72 hours post-transfection and treatment with GDC-0941. B. Validation of decreased levels of DDN mRNA following transfection of MCF-7 cells with DDN-targeting siRNA or non-silencing siRNA, in the presence or absence of 300 nM GDC-0941. Plotted is expression of DDN mRNA relative to expression of β-actin mRNA, as determined by qRT-PCR. C-E. Quantitative phosphorylation analysis of MCF-7 cells transfected with DDN-targeting siRNA or non-silencing siRNA, in the presence or absence of 300 nM GDC-0941 for 24 hours. Cells were treated with treated with 300 nM GDC-0941 or DMSO control for 24 hours, as indicated, with phospho-specific epitopes indicated above each graph. F. MDA-MB-231 cells transfected with DDN-targeting siRNA or non-silencing siRNA, in the presence or absence of 1 μM GDC-0941. Cell viability was assayed at 0, 24, 48, and 72 hours post-transfection and treatment with GDC-0941. G. Validation of decreased levels of DDN mRNA following transfection of MDA-MB-231 cells with DDN-targeting siRNA or non-silencing siRNA, in the presence or absence of 1 μM GDC-0941. Plotted is expression of DDN mRNA relative to expression of β-actin mRNA, as determined by qRT-PCR. H-J. Quantitative phosphorylation analysis of MDA-MB-231 cells transfected with DDN-targeting siRNA or non-silencing siRNA, in the presence or absence of 1 μM GDC-0941 for 24 hours. Cells were treated with treated with 1 μM GDC-0941 or DMSO control for 24 hours, as indicated, with phospho-specific epitopes indicated above each graph. Data in panels B and G represent mean±SD (n=3). Data in panels C-D and H-J represent mean±SEM (n=3);*, P<0.05;**, P<0.01;***, P<0.001.

FIG. 14 shows quantitative phosphorylation analysis of MCF-7 cells transfected with DDN-targeting siRNA or non-silencing siRNA, in the presence or absence of 300 nM GDC-0941 for 24 hours. Cells were treated with 300 nM GDC-0941 or DMSO control for 24 hours, as indicated, with phospho-specific epitopes indicated above each graph. All of the above data represent mean±SEM (n=3);*, P<0.05;**, P<0.01;***, P<0.001.

FIG. 15 shows quantitative phosphorylation analysis of MDA-MB-231 cells transfected with DDN-targeting siRNA or non-silencing siRNA, in the presence or absence of 1 μM GDC-0941 for 24 hours. Cells were treated with treated with 1 μM GDC-0941 or DMSO control for 24 hours, as indicated, with phospho-specific epitopes indicated above each graph. All of the above data represent mean±SEM (n=3);*, P<0.05;**, P<0.01;***, P<0.001.

### DETAILED DESCRIPTION

### Definitions

Unless defined otherwise, technical and scientific terms used herein have the same meaning as commonly understood by one of ordinary skill in the art to which this invention belongs. Singleton et al., Dictionary of Microbiology and Molecular Biology, 2^{nd} ed., J. Wiley & Sons (New York, N.Y. 1994), and March, Advanced Organic Chemistry Reactions, Mechanisms and Structure, 4^{th} ed., J. Wiley & Sons (New York, NY 1992), provide one skilled in the art with a general guide to many of the terms used in the present application.

One skilled in the art will recognize many methods and materials similar or equivalent to those described herein that may be used in the practice of the present invention. Indeed, the present invention is in no way limited to the methods and materials described herein. For purposes of the invention, the following terms are defined below.

As used here, the term “activator of a gene” refers to any molecule, compound, chemical, polypeptide, protein, or nucleic acid that increases the expression level or activity of a gene or its expression product. The term “inhibitor of a gene” as used herein refers to any molecule, compound, chemical, polypeptide, protein, or nucleic acid that reduces the expression level or activity of a gene or its expression product. Examples of inhibitors include small interfering RNAs (siRNAs), antisense RNAs, ribozymes, monoclonal antibodies, and polyclonal antibodies.

As used herein, the term “amplicon” refers to a piece of DNA that has been synthesized using an amplification technique, such as the polymerase chain reaction (PCR) and ligase chain reaction.

The term “anti-cancer agent” as used herein refers to any molecule, compound, chemical, or composition that has an anti-cancer effect. Anti-cancer agents include, without limitation, chemotherapeutic agents, radiotherapeutic agents, cytokines, anti-angiogenic agents, apoptosis-inducing agents or anti-cancer immunotoxins, such as antibodies. Examples of anti-cancer agents include, without limitation, bevacizumab, trastuzumab, erlotinib, methotrexate, taxol, mercaptopurine, thioguanine, hydroxyurea, cytarabine, cyclophosphamide, ifosfamide, nitrosoureas, mitomycin, dacarbazine, procarbizine, etoposides, campathecins, bleomycin, doxorubicin, idarubicin, daunorubicin, dactinomycin, plicamycin, mitoxantrone, asparaginase, vinblastine, vincristine, vinorelbine, paclitaxel, docetaxel, fluorouracil (5-FU), and leucovorin. Other anti-cancer agents are known in the art. In an embodiment of the invention, the anti-cancer agent is selected from bevacizumab, trastuzumab, and erlotinib.

The terms “assay” or “assaying” as used herein refer to performing a quantitative or qualitative analysis of a component in a sample. The terms include laboratory or clinical observations, and/or measuring the level of the component in the sample.

The terms “cancer” and “cancerous” as used herein, refer to or describe the physiological condition that is typically characterized by unregulated cell growth. Examples of cancer in the present application include cancers of epithelial origin, including breast cancer, colorectal cancer, non-small cell lung cancer, renal cancer, ovarian cancer, prostate cancer, and melanoma.

The term “co-expressed” as used herein refers to a statistical correlation between the expression level of one gene and the expression level of another gene. Pairwise co-expression may be calculated by various methods known in the art, e.g., by calculating Pearson correlation coefficients or Spearman correlation coefficient. Co-expressed gene cliques may also be identified using a graph theory. An analysis of co-expression may be calculated using normalized expression data.

The term “correlates” or “correlating” as used herein refers to a statistical association between instances of two events, where events may include numbers, data sets, and the like. For example, when the events involve numbers, a positive correlation (also referred to herein as a “direct correlation”) means that as one increases, the other increases as well. A negative correlation (also referred to herein as an “inverse correlation”) means that as one increases, the other decreases. The present invention provides genes, the expression levels of which are correlated with a particular outcome measure, such as between the expression level of a gene and the likelihood of sensitivity to a PI3K inhibitor. For example, the increased expression level of a gene product may be positively correlated with a likelihood of sensitivity to a PI3K inhibitor in a tumor cell, tumor, or patient. Such a positive correlation may be demonstrated statistically in various ways, e.g. by a low hazard ratio. In another example, the increased expression level of a gene product may be positively correlated with a likelihood of resistance to a PI3K inhibitor. Such a negative correlation may be demonstrated statistically in various ways, e.g., a high hazard ratio.

The term “Ct” as used herein refers to threshold cycle, the cycle number in quantitative polymerase chain reaction (qPCR) at which the fluorescence generated within a reaction well exceeds the defined threshold, i.e. the point during the reaction at which a sufficient number of amplicons have accumulated to meet the defined threshold.

The term “expression level” as used herein refers to qualitative or quantitative determination of an expression product or gene product. Expression level may be determined for the RNA expression level of a gene or for the polypeptide expression level of a gene. The term “normalized” expression level as used herein refers to an expression level of a response indicator gene relative to the level of an expression product of a reference gene(s), which might be all measured expression products in the sample, a single reference expression product, or a particular set of expression products. A gene exhibits an “increased expression level” when the expression level of an expression product is higher in a first sample, such as in a PI3K inhibitor-sensitive tumor cell, than in a second sample, such as in a PI3K inhibitor-resistant tumor cell. In an embodiment, the first sample may be a clinically relevant subpopulation of patients (e.g., patients who are sensitive to a PI3K inhibitor), and the second sample may be a related subpopulation (e.g., patients who are resistant to the PI3K inhibitor). Similarly, a gene exhibits an “increased normalized expression level” when the normalized expression level of an expression product is higher in a first sample than in a second sample.

The term “expression product” or “gene product” are used herein to refer to the RNA transcription products (transcripts) of a gene, including mRNA, and the polypeptide translation products of such RNA transcripts. An expression product may be, for example, an unspliced RNA, an mRNA, a splice variant mRNA, a microRNA, a fragmented RNA, a polypeptide, a post-translationally modified polypeptide, a splice variant polypeptide, etc.

The term “measuring” as used herein refers to performing a physical act of determining the dimension, quantity, or capacity of a component in a sample.

The term “microarray” as used herein refers to an ordered arrangement of hybridizable array elements, e.g., oligonucleotide or polynucleetide probes, on a substrate.

The term “polynucleotide” generally refers to any polyribonucleotide or polydeoxribonucleotide, which may be unmodified RNA or DNA or modified RNA or DNA. Thus, for instance, polynucleotides as used herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” also includes DNAs (including cDNAs) and RNAs and those that contain one or more modified bases. Thus, DNAs or RNAs with backbones modified for stability or for other reasons, are “polynucleotides” as that term is used herein. Moreover, DNAs or RNAs comprising unusual bases, such as inosine, or modified bases, such as tritiated bases, are included within the term “polynucleotides” as used herein. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “oligonucleotide” refers to a relatively short polynucleotide, including, without limitation, single-stranded deoxyribonucleotides, single- or double-stranded ribonucleotides, RNA/DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

The term “primer” or “oligonucleotide primer” as used herein, refers to an oligonucleotide that acts to initiate synthesis of a complementary nucleic acid strand when placed under conditions in which synthesis of a primer extension product is induced, e.g., in the presence of nucleotides and a polymerization-inducing agent such as a DNA or RNA polymerase and at suitable temperature, pH, metal ion concentration, and salt concentration. Primers are generally of a length compatible with their use in synthesis of primer extension products, and can be in the range of between about 8 nucleotides and about 100 nucleotides (nt) in length, such as about 10 nt to about 75 nt, about 15 nt to about 60 nt, about 15 nt to about 40 nt, about 18 nt to about 30 nt, about 20 nt to about 40 nt, about 21 nt to about 50 nt, about 22 nt to about 45 nt, about 25 nt to about 40 nt, and so on, e.g., in the range of between about 18 nt and about 40 nt, between about 20 nt and about 35 nt, between about 21 and about 30 nt in length, inclusive, and any length between the stated ranges. Primers can be in the range of between about 10-50 nucleotides long, such as about 15-45, about 18-40, about 20-30, about 21-25 nt and so on, and any length between the stated ranges. In some embodiments, the primers are not more than about 10, 12, 15, 20, 21, 22, 23, 24, 25, 26, 27, 28, 29, 30, 35, 40, 45, 50, 55, 60, 65, or 70 nucleotides in length. In this context, the term “about” may be construed to mean 1, 2, 3, 4, 5, 6, 7, 8, 9, 10, 11, 12, 13, 14, 15, 16, 17, 18, 19 or 20 more nucleotides either 5′ or 3′ from either termini or from both termini.

Primers are in many embodiments single-stranded for maximum efficiency in amplification, but may alternatively be double-stranded. If double-stranded, the primer is in many embodiments first treated to separate its strands before being used to prepare extension products. This denaturation step is typically effected by heat, but may alternatively be carried out using alkali, followed by neutralization. Thus, a “primer” is complementary to a template, and complexes by hydrogen bonding or hybridization with the template to give a primer/template complex for initiation of synthesis by a polymerase, which is extended by the covalent addition of bases at its 3′ end.

A “primer pair” as used herein refers to first and second primers having nucleic acid sequence suitable for nucleic acid-based amplification of a target nucleic acid. Such primer pairs generally include a first primer having a sequence that is the same or similar to that of a first portion of a target nucleic acid, and a second primer having a sequence that is complementary to a second portion of a target nucleic acid to provide for amplification of the target nucleic acid or a fragment thereof. Reference to “first” and “second” primers herein is arbitrary, unless specifically indicated otherwise. For example, the first primer can be designed as a “forward primer” (which initiates nucleic acid synthesis from a 5′ end of the target nucleic acid) or as a “reverse primer” (which initiates nucleic acid synthesis from a 5′ end of the extension product produced from synthesis initiated from the forward primer). Likewise, the second primer can be designed as a forward primer or a reverse primer.

As used herein, the term “probe” or “oligonucleotide probe”, used interchangeably herein, refers to a structure comprised of a polynucleotide, as defined above, that contains a nucleic acid sequence complementary to a nucleic acid sequence present in the target nucleic acid analyte (e.g., a nucleic acid amplification product). The polynucleotide regions of probes may be composed of DNA, and/or RNA, and/or synthetic nucleotide analogs. Probes are generally of a length compatible with their use in specific detection of all or a portion of a target sequence of a target nucleic acid, and are in many embodiments in the range of between about 8 nt and about 100 nt in length, such as about 8 to about 75 nt, about 10 to about 74 nt, about 12 to about 72 nt, about 15 to about 60 nt, about 15 to about 40 nt, about 18 to about 30 nt, about 20 to about 40 nt, about 21 to about 50 nt, about 22 to about 45 nt, about 25 to about 40 nt in length, and so on, e.g., in the range of between about 18-40 nt, about 20-35 nt, or about 21-30 nt in length, and any length between the stated ranges. In some embodiments, a probe is in the range of between about 10-50 nucleotides long, such as about 15-45, about 18-40, about 20-30, about 21-28, about 22-25 and so on, and any length between the stated ranges. In some embodiments, the probes are not more than about 10, 12, 15, 20, 21, 22, 23, 24, 25, 26, 27, 28, 29, 30, 35, 40, 45, 50, 55, 60, 65, or 70 nucleotides in length. In this context, the term “about” may be construed to mean 1, 2, 3, 4, 5, 6, 7, 8, 9, 10, 11, 12, 13, 14, 15, 16, 17, 18, 19 or 20 more nucleotides either 5′ or 3′ from either termini or from both termini.

The term “phosphoinositide 3-kinase (PI3K) inhibitor” as used herein refers to a molecule or a composition comprising a molecule that functions by inhibiting a phospoinositide 3-kinase enzyme, which is part of the PI3K/AKT/mTOR pathway. Examples of PI3K inhibitors include, but are not limited to, perifosine, CAL101, PX-866, BEZ235, SF1126, INK111, IPI-145, GDC-0941, BKM120, XL1417, XL765, Palomid 529, ZSTK474, and PWT33597. In an embodiment of the invention, the PI3K inhibitor is GDC-0941.

The PI3K inhibitor may be used alone, or in combination with other anti-cancer agents. For example, GDC-0941 may be used alone, or in combination with bevacizumab, trastuzumab, or erlotinib.

The term “sensitive to a PI3K inhibitor” as used herein refers to a favorable response to a PI3K inhibitor as opposed to an unfavorable response, such as adverse events. A positive response reflecting sensitivity to a PI3K inhibitor may include, without limitation, (1) inhibition, to some extent, of tumor cell growth or tumor growth, including slowing down to complete growth arrest; (2) reduction in the number of tumor cells; (3) reduction in tumor size; (4) inhibition (i.e., reduction, slowing down or complete cessation) of tumor cell infiltration into adjacent peripheral organs and/or tissues; (5) inhibition of metastasis; (6) enhancement of anti-tumor immune response, possibly resulting in regression or rejection of the tumor; (7) relief, to some extent, of one or more symptoms associated with the tumor; (8) increase in the length of survival following treatment; and/or (9) decreased mortality at a given point of time following treatment. In individual patients, a positive response can be expressed in terms of a number of clinical parameters, including loss of detectable tumor (complete response, CR), decrease in tumor size and/or cancer cell number (partial response, PR), tumor growth arrest (stable disease, SD), enhancement of anti-tumor immune response, possibly resulting in regression or rejection of the tumor, relief, to some extent, of one or more symptoms associated with the tumor, increase in the length of survival following treatment; and/or decreased mortality at a given point of time following treatment. Continued increase in tumor size and/or cancer cell number and/or tumor metastasis is indicative of resistance to the PI3K inhibitor. Thus, the term “resistant to a PI3K inhibitor” as used herein refers to no change in the state of a tumor cell, tumor, or patient upon contact or treatment with a PI3K inhibitor, or to an unfavorable response. An unfavorable response may include a response that is opposite that of the positive responses described above. An unfavorable response may also include an undesirable toxic side effect of the PI3K inhibitor.

In a patient, sensitivity to a PI3K inhibitor can be evaluated on the basis of one or more endpoints. For example, analysis of overall response rate (ORR) classifies as responders those patients who experience CR or PR after treatment with a PI3K inhibitor. Analysis of disease control (DC) classifies as responders those patients who experience CR, PR or SD after treatment with a PI3K inhibitor.

The term “prediction” is used herein to refer to the likelihood that a cancer cell or a cancer patient will have a particular response to treatment, whether positive or negative. In the context of a cancer patient, “prediction” refers to a particular response to treatment following surgical removal of the primary tumor. For example, treatment could include treatment with a PI3K inhibitor.

The predictive methods of the present invention may be used clinically to make treatment decisions by choosing the most appropriate treatment modalities for any particular patient. The predictive methods of the present invention are useful tools in predicting if a patient is likely to exhibit sensitivity to a PI3K inhibitor, surgical intervention, or both.

The term “reference gene” as used herein refers to a gene whose expression level can be used to compare the expression level of a gene product in a test sample. In an embodiment of the invention, reference genes include housekeeping genes, such as beta-globin, alcohol dehydrogenase, or any other gene, the expression of which does not vary depending on the disease status of the cell containing the gene. In another embodiment, all of the assayed genes or a large subset thereof may serve as reference genes.

The term “response indicator gene” as used herein refers to a gene, the expression of which correlates positively with sensitivity or resistance to a PI3K inhibitor, such as GDC-0941. The expression of a response indicator gene may be determined by assaying or measuring the expression level of an expression product of the response indicator gene.

The term “RNA transcript” as used herein refers to the RNA transcription product of a gene, including, for example, mRNA, an unspliced RNA, a splice variant mRNA, a microRNA, and a fragmented RNA.

Unless indicated otherwise, each gene name used herein corresponds to the Official Symbol assigned to the gene and provided by Entrez Gene (URL: www.ncbi.nlm.nih.gov/sites/entrez) as of the filing date of this application.

The term “tumor cell” as used herein refers to a cancerous cell obtained from a cancer cell line or a cancer patient. The term encompasses a tumor cell obtained from tumor tissue samples, for example, tissue obtained by surgical resection and tissue obtained by biopsy, such as for example, a core biopsy or a fine needle biopsy. The term “tumor cell” also encompasses tumor cells obtained from sites other than the primary tumor, e.g., circulating tumor cells. The term further encompasses cells that are the progeny of the patient's tumor cells, e.g. cell culture samples derived from primary tumor cells or circulating tumor cells. The term further encompasses samples that may comprise protein or nucleic acid material shed from tumor cells in vivo, e.g., bone marrow, blood, plasma, serum, and the like. The term also encompasses samples that have been enriched for tumor cells or otherwise manipulated after their procurement and samples comprising polynucleotides and/or polypeptides that are obtained from a patient's tumor material.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to re-anneal when complementary strands are present in an environment below their melting temperature. The higher the degree of desired homology between the probe and hybridizable sequence, the higher the relative temperature that can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, typically: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide, followed by a high-stringency wash consisting of 0.1×SSC containing EDTA at 55° C.

“Moderately stringent conditions” may be identified as described by Sambrook et al., Molecular Cloning: A Laboratory Manual, New York: Cold Spring Harbor Press, 1989, and include the use of washing solution and hybridization conditions (e.g., temperature, ionic strength and %SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

The terms “subject,” “individual,” and “patient” are used interchangeably herein to refer to a mammal being assessed for treatment and/or being treated. In an embodiment, the mammal is a human. The terms “subject,” “individual,” and “patient” thus encompass individuals having cancer (e.g., colorectal cancer or other cancer referenced herein), including those who have undergone or are candidates for resection (surgery) to remove cancerous tissue (e.g., cancerous colorectal tissue or other cancer referenced herein).

The terms “treatment” and “treating” refer to administering or contacting an agent, or carrying out a procedure (e.g., radiation, a surgical procedure, etc.), for the purpose of obtaining an effect. In a subject, the effect may be prophylactic in terms of completely or partially preventing a disease or symptom thereof and/or may be therapeutic in terms of effecting a partial or complete cure for a disease and/or symptoms of the disease. The terms cover any treatment of a disease in a mammal, particularly in a human, and includes: (a) preventing the disease or a symptom of a disease from occurring in a subject that may be predisposed to the disease but has not yet been diagnosed as having it (e.g., including diseases that may be associated with or caused by a primary disease); (b) inhibiting the disease, i.e., arresting its development; and (c) relieving the disease, i.e., causing regression of the disease.

The term “tumor” as used herein, refers to all neoplastic cell growth and proliferation, whether malignant or benign, and all pre-cancerous and cancerous cells and tissues.

The term “computer-based system”, as used herein refers to the hardware means, software means, and data storage means used to analyze information. The minimum hardware of a patient computer-based system comprises a central processing unit (CPU), input means, output means, and data storage means. A skilled artisan can readily appreciate that many of the currently available computer-based system are suitable for use in the present invention and may be programmed to perform the specific measurement and/or calculation functions of the present invention.

To “record” data, programming or other information on a computer readable medium refers to a process for storing information, using any such methods as known in the art. Any convenient data storage structure may be chosen, based on the means used to access the stored information. A variety of data processor programs and formats can be used for storage, e.g. word processing text file, database format, etc.

A “processor” or “computing means” references any hardware and/or software combination that will perform the functions required of it. For example, any processor herein may be a programmable digital microprocessor such as available in the form of an electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

Before the present invention and specific exemplary embodiments of the invention are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

As used herein and in the appended claims, the singular forms “a,” “an,” and “the” include plural referents unless the context clearly dictates otherwise. Thus, for example, reference to “a tumor cell” includes a plurality of such tumor cells and reference to “a PI3K inhibitor” includes reference to one or more PI3K inhibitors, and so forth.

### DETAILED DESCRIPTION

The practice of the methods and compositions of the present disclosure will employ, unless otherwise indicated, conventional techniques of molecular biology (including recombinant techniques), microbiology, cell biology, and biochemistry, which are within the skill of the art. Such techniques are explained fully in the literature, such as, “Molecular Cloning: A Laboratory Manual”, 2nd edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology”, 4th edition (D. M. Weir & C. C. Blackwell, eds., Blackwell Science Inc., 1987); “Gene Transfer Vectors for Mammalian Cells” (J. M. Miller & M. P. Calos, eds., 1987); “Current Protocols in Molecular Biology” (F. M. Ausubel et al., eds., 1987); and “PCR: The Polymerase Chain Reaction”, (Mullis et al., eds., 1994).

The present invention provides response indicator genes of PI3K inhibitors. These genes are listed in Tables 2-4. The present invention provides a number of methods that utilize the response indicator genes and associated information. In a first aspect, the present invention provides a method of determining whether a tumor cell is likely to be sensitive or resistant to a PI3K inhibitor. In another aspect, the present invention provides a method of predicting a likelihood that a patient with cancer will be sensitive or resistant to a treatment comprising a PI3K inhibitor. The methods of the invention comprise assaying or measuring the expression level of the response indicator gene(s) in a tumor cell or a sample comprising tumor cells, and determining the likelihood that the tumor cell or the patient will be sensitive or resistant to the PI3K inhibitor based on the correlation between the expression level of the response indicator gene(s) and sensitivity or resistance to the PI3K inhibitor.

The response indicator genes and associated information provided by the present invention also have utility in the development of therapies to treat cancers and screening patients for inclusion in clinical trials that test the efficacy of PI3K inhibitor. The response indicator genes and associated information may further be used to design or produce a reagent that modulates the level or activity of the expression product. Such reagents may include, but are not limited to, an antisense RNA, a small inhibitory RNA (siRNA), a ribozyme, a small molecule, a monoclonal antibody, and a polyclonal antibody. Thus, for example, the present invention provides a method for increasing sensitivity of a tumor cell (or a cancer patient) to a PI3K inhibitor by contacting the tumor cell with (or administering to the patient) an inhibitor of one or more response indicator genes selected from Table 3, and contacting the tumor cell with (or administering to the patient) a PI3K inhibitor. The present invention also provides a method for increasing sensitivity of a tumor cell (or a cancer patient) to a PI3K inhibitor by contacting the tumor cell with (or administering to the patient) an activator of one or more response indicator genes selected from Table 2, and contacting the tumor cell with (or administering to the patient) a PI3K inhibitor.

In various embodiments of the methods of the present invention, various technological approaches are available for assaying or measuring the expression levels of the response indicator genes, including, without limitation, RT-PCR, microarrays, serial analysis of gene expression (SAGE), and nucleic acid sequencing, which are described in more detail below.

### Methods to Predict Likelihood of Sensitivity or Resistance to a PI3K Inhibitor

As described above, a number of response indicator genes were identified. Expression levels or normalized expression levels of these indicator gene products can then be determined in tumor cells. The response indicator genes are useful for predicting the likelihood that a tumor cell will be sensitive or resistant to a PI3K inhibitor. The response indicator genes are also useful for predicting the likelihood that a cancer patient will be sensitive or resistant to treatment with a PI3K inhibitor. In such methods, the expression level or normalized expression level of one or more response indicator genes are determined in a tumor sample obtained from an individual patient who has cancer and for whom treatment with a PI3K inhibitor is being contemplated. Depending on the outcome of the assessment, treatment with a PI3K inhibitor may be indicated, or an alternative treatment regimen may be indicated.

In carrying out the method of the present invention, tumor cells or a tumor sample is assayed or measured for an expression level of a response indicator gene product(s). The tumor sample can be obtained from a solid tumor, e.g., via biopsy, or from a surgical procedure carried out to remove a tumor; or from a tissue or bodily fluid that contains cancer cells. In an embodiment of the invention, the tumor cell or tumor sample is obtained from a tumor of epithelial origin. In another embodiment of the invention, the tumor cell is a breast, colon, non-small cell lung, renal, ovarian, prostate, or melanoma tumor cell. In another embodiment, the tumor sample is obtained from a patient with breast cancer, colon cancer, non-small cell lung cancer, renal cancer, ovarian cancer, prostate cancer, or melanoma. In another embodiment, the expression level of a response indicator gene is normalized relative to the level of an expression product of one or more reference genes. In a particular embodiment of the invention, the PI3K inhibitor is GDC-0941.

The likelihood of sensitivity or resistance to a PI3K inhibitor is predicted by comparing, directly or indirectly, the expression level or normalized expression level of the response indicator gene in the tumor cell or tumor sample from an individual patient to the expression level or normalized expression level of the response indicator gene in a relevant cell population or clinically relevant subpopulation of patients. Thus, as explained above, when the response indicator gene analyzed is a gene that shows increased expression in a tumor cell population sensitive to a PI3K inhibitor compared to its expression in a tumor cell population resistant to the PI3K inhibitor, then if the expression level of the gene in the tumor cell being analyzed trends toward a level of expression characteristic of a sensitive cell, then the gene expression level supports a determination that the tumor cell is more likely to be sensitive to the PI3K inhibitor. Similarly, where the response indicator gene analyzed is a gene that is increased in expression in a cancer patient population sensitive to a PI3K inhibitor as compared to a cancer patient population resistant to the PI3K inhibitor, then if the expression level of the gene in the individual subject trends toward a level of expression characteristic of a patient sensitive to the PI3K inhibitor, then the gene expression level supports a determination that the individual patient will more likely be sensitive to the PI3K inhibitor.

It is understood that the expression level or normalized expression level of a response indicator gene from a tumor cell or a tumor sample from an individual patient can be compared, directly or indirectly, to the expression level or normalized expression level of the response indicator gene in a relevant cell population or in a clinically relevant subpopulation of patients. For example, when compared indirectly, the expression level or normalized expression level of the response indicator gene from an individual patient may be used to calculate a quantitative score indicating the likelihood of sensitivity to a PI3K inhibitor, such as a treatment score (TS) as described below, and compared to a calculated quantitative score in the clinically relevant subpopulation of patients.

It is also understood that it can be useful to measure the expression level of a response indicator gene product at multiple time points, for example, prior to and during the course of treatment with a PI3K inhibitor. For example, an initial assessment of the likelihood that a patient will respond to treatment with PI3K inhibitor can be made prior to initiation of treatment in order to optimize treatment choice.

Development of drug resistance is a well-known phenomenon in treatment of cancer patients. As they proliferate, tumor cells can accumulate mutations that confer drug resistance through a variety of mechanisms, including resistance to a PI3K inhibitor. Tests that utilize the measurement of response indicator genes to assess the likelihood of sensitivity or resistant to a PI3K inhibitor can be carried out at time intervals to monitor changes indicative of the onset of drug resistance that may arise from changes in the tumor over time. It is not necessary to know what mutations or changes have taken place in the tumor in order to monitor consequent changes in the gene expression level of response indicator genes and assess the likelihood of continuing sensitivity to the PI3K inhibitor.

Upon determination that a tumor cell or a patient has a low likelihood of sensitivity to a PI3K inhibitor, it may be desirable to increase sensitivity of the tumor cell or the patient to the PI3K inhibitor. Thus, if a tumor cell or a tumor sample from a patient exhibits low expression of one or more genes selected from Table 2, sensitivity to the PI3K inhibitor may be increased by contacting the tumor cell, or administering to the patient, an activator of the gene(s). If a tumor cell or a tumor sample from a patient exhibits high expression of one or more genes selected from Table 3, sensitivity to the PI3K inhibitor may be increased by contacting the tumor cell, or administering to the patient, an inhibitor of the gene(s).

### Correlating Expression Level of a Response Indicator Gene Product to Sensitivity or Resistance to a PI3K Inhibitor

Many statistical methods may be used to determine if there is a correlation between expression levels of response indicator genes and sensitivity or resistance to a PI3K inhibitor. For example, this relationship can be presented as a continuous treatment score (TS), or they may be stratified into sensitivity or benefit groups (e.g., low, intermediate, high). The interaction studied may vary, e.g. standard of care vs. new treatment, or surgery alone vs. surgery followed by treatment with a PI3K inhibitor. For example, a Cox proportional hazards regression could be used to model the follow-up data, i.e. censoring time to recurrence at a certain time (e.g., 3 years) after randomization for patients who have not experienced a recurrence before that time, to determine if the TS is associated with the magnitude of benefit from the PI3K inhibitor. One might use the likelihood ratio test to compare the reduced model with RS, TS and the treatment main effect, with the full model that includes RS, TS, the treatment main effect, and the interaction of treatment and TS. A pre-determined p-value cut-off (e.g., p<0.05) may be used to determine significance. In an exemplary embodiment, power calculations are carried out for the Cox proportional hazards model with a single non-binary covariate using the method proposed by F. Hsieh and P. Lavori, Control Clin Trials 21:552-560 (2000) as implemented in PASS 2008.

The response indicator genes of PI3K inhibitors of the present invention are listed in Tables 2-4. In an embodiment of the invention, increased expression level of one or more genes selected from Table 2 is positively correlated with a likelihood of sensitivity to a PI3K inhibitor. In another embodiment of the invention, increased expression level of one or more genes selected from Table 3 positively correlated with a likelihood of resistance to a PI3K inhibitor.

In a specific embodiment of the invention, increased expression level of one or more genes selected from VAV3, DGKE, MBD1, SERP1, TXNL1, NLGN4X, C1ORF91, SLC25A1, ZBTB40, FAM51A1, LOC116349, KIAA1468, PIGN, PKD1L1, SELT, CISH, MGC50559, NPY5R, PEX10, and C6ORF35 is positively correlated with a likelihood of sensitivity to a PI3K inhibitor, and increased expression level of one or more genes selected from TRIM50C, GALR2, INSL3, LOC389633, GTPBP10, PGRMC1, DNASE1L3, CACNG2, FAM90A1, OGT, FKBP6, GDAP1L1, CHRNB1, NLGN3, ZNF259, DDN, NXF3, MGC35366, TANK, and LOC116123 is positively correlated with a likelihood of resistance to a PI3K inhibitor.

In a particular embodiment of the invention, the PI3K inhibitor is GDC-0941 and the response indicator gene(s) is assayed or measured in tumor cells of epithelial origin, such as breast, colon, non-small cell lung, renal, ovarian, prostate, and melanoma tumor cells. The tumor cells may be derived from a tumor sample obtained from a human patient with cancer. In another embodiment, the expression level of the response indicator gene(s) is normalized as described in more detail below.

### Methods of Assaying Expression Levels of a Gene Product

The methods and compositions of the present disclosure will employ, unless otherwise indicated, conventional techniques of molecular biology (including recombinant techniques), microbiology, cell biology, and biochemistry, which are within the skill of the art. Exemplary techniques are explained in the literature, such as, “Molecular Cloning: A Laboratory Manual”, 2^{th} edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology”, 4^{th} edition (D. M. Weir & C. C. Blackwell, eds., Blackwell Science Inc., 1987); “Gene Transfer Vectors for Mammalian Cells” (J. M. Miller & M. P. Calos, eds., 1987); “Current Protocols in Molecular Biology” (F. M. Ausubel et al., eds., 1987); and “PCR: The Polymerase Chain Reaction” (Mullis et al., eds., 1994).

Methods of gene expression profiling include methods based on hybridization analysis of polynucleotides, methods based on sequencing of polynucleotides, and proteomics-based methods. Exemplary methods known in the art for the quantification of mRNA expression in a sample include northern blotting and in situ hybridization (Parker & Barnes, Methods in Molecular Biology 106:247-283 (1999)); RNAse protection assays (Hod, Biotechniques 13:852-854 (1992)); and PCR-based methods, such as reverse transcription PCT (RT-PCR) (Weis et al., Trends in Genetics 8:263-264 (1992)). Antibodies may be employed that can recognize sequence-specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS).

### Reverse Transcriptase PCR (RT-PCR)

Typically, mRNA is isolated from a sample. The starting material is typically total RNA isolated from a human tumor, usually from a primary tumor. Optionally, normal tissues from the same patient can be used as an internal control. mRNA can be extracted from a tissue sample, e.g., from a sample that is fresh, frozen (e.g. fresh frozen), or paraffin-embedded and fixed (e.g. formalin-fixed).

General methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56:A67 (1987), and De Andrés et al., BioTechniques 18:42044 (1995). In particular, RNA isolation can be performed using a purification kit, buffer set and protease from commercial manufacturers, such as Qiagen, according to the manufacturer's instructions. For example, total RNA from cells in culture can be isolated using Qiagen RNeasy mini-columns. Other commercially available RNA isolation kits include MasterPure™ Complete DNA and RNA Purification Kit (EPICENTRE®, Madison, Wis.), and Paraffin Block RNA Isolation Kit (Ambion, Inc.). Total RNA from tissue samples can be isolated using RNA Stat-60 (Tel-Test). RNA prepared from a tumor sample can be isolated, for example, by cesium chloride density gradient centrifugation.

The sample containing the RNA is then subjected to reverse transcription to produce cDNA from the RNA template, followed by exponential amplification in a PCR reaction. The two most commonly used reverse transcriptases are avian myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

PCR-based methods use a thermostable DNA-dependent DNA polymerase, such as a Taq DNA polymerase. For example, TaqMan® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction product. A third oligonucleotide, or probe, can be designed to facilitate detection of a nucleotide sequence of the amplicon located between the hybridization sites of the two PCR primers. The probe can be detectably labeled, e.g., with a reporter dye, and can further be provided with both a fluorescent dye, and a quencher fluorescent dye, as in a Taqman® probe configuration. Where a Taqman® probe is used, during the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TaqMan® RT-PCR can be performed using commercially available equipment, such as, for example, ABI PRISM 7700™ Sequence Detection System™ (Perkin-Elmer-Applied Biosystems, Foster City, Calif., USA), or Lightcycler (Roche Molecular Biochemicals, Mannheim, Germany). In a preferred embodiment, the 5′ nuclease procedure is run on a real-time quantitative PCR device such as the ABI PRISM 7700™ Sequence Detection System™. The system consists of a thermocycler, laser, charge-coupled device (CCD), camera and computer. The system amplifies samples in a 384-well format on a thermocycler. The RT-PCR may be performed in triplicate wells with an equivalent of 2 ng RNA input per 10 μL-reaction volume. During amplification, laser-induced fluorescent signal is collected in real-time through fiber optics cables for all wells, and detected at the CCD. The system includes software for running the instrument and for analyzing the data.

5′-Nuclease assay data are generally initially expressed as a threshold cycle (“Cₜ”). Fluorescence values are recorded during every cycle and represent the amount of product amplified to that point in the amplification reaction. The threshold cycle (Cₜ) is generally described as the point when the fluorescent signal is first recorded as statistically significant.

To minimize errors and the effect of sample-to-sample variation, RT-PCR is usually performed using an internal standard. The ideal internal standard gene (also referred to as a reference gene) is expressed at a constant level among cancerous and non-cancerous tissue of the same origin (i.e., a level that is not significantly different among normal and cancerous tissues), and is not significantly affected by the experimental treatment (i.e., does not exhibit a significant difference in expression level in the relevant tissue as a result of exposure to chemotherapy). For example, reference genes useful in the methods disclosed herein should not exhibit significantly different expression levels in cancerous colon as compared to normal colon tissue. RNAs most frequently used to normalize patterns of gene expression are mRNAs for the housekeeping genes glyceraldehyde-3-phosphate-dehydrogenase (GAPDH) and β-actin. Exemplary reference genes used for normalization comprise one or more of the following genes: ATP5E, GPX1, PGK1, UBB, and VDAC2. Gene expression measurements can be normalized relative to the mean of one or more (e.g., 2, 3, 4, 5, or more) reference genes. Reference-normalized expression measurements can range from 0 to 15, where a one unit increase generally reflects a 2-fold increase in RNA quantity.

Real time PCR is compatible both with quantitative competitive PCR, where an internal competitor for each target sequence is used for normalization, and with quantitative comparative PCR using a normalization gene contained within the sample, or a housekeeping gene for RT-PCR. For further details see, e.g. Held et al., Genome Research 6:986-994 (1996).

The steps of a representative protocol for use in the methods of the present disclosure use fixed, paraffin-embedded tissues as the RNA source. mRNA isolation, purification, primer extension and amplification can be preformed according to methods available in the art. (see, e.g., Godfrey et al. J. Molec. Diagnostics 2: 84-91 (2000); Specht et al., Am. J. Pathol. 158: 419-29 (2001)). Briefly, a representative process starts with cutting about 10 μm thick sections of paraffin-embedded tumor tissue samples. The RNA is then extracted, and protein and DNA are depleted from the RNA-containing sample. After analysis of the RNA concentration, RNA is reverse transcribed using gene specific primers followed by RT-PCR to provide for cDNA amplification products.

### Design of PCR Primers and Probes

PCR primers and probes can be designed based upon exon or intron sequences present in the mRNA transcript of the gene of interest. Primer/probe design can be performed using publicly available software, such as the DNA BLAT software developed by Kent, W. J., Genome Res. 12(4):656-64 (2002), or by the BLAST software including its variations.

Where necessary or desired, repetitive sequences of the target sequence can be masked to mitigate non-specific signals. Exemplary tools to accomplish this include the Repeat Masker program available on-line through the Baylor College of Medicine, which screens DNA sequences against a library of repetitive elements and returns a query sequence in which the repetitive elements are masked. The masked sequences can then be used to design primer and probe sequences using any commercially or otherwise publicly available primer/probe design packages, such as Primer Express (Applied Biosystems); MGB assay-by-design (Applied Biosystems); Primer3 (Steve Rozen and Helen J. Skaletsky (2000) Primer3 on the WWW for general users and for biologist programmers. In: Rrawetz S, Misener S (eds) Bioinformatics Methods and Protocols: Methods in Molecular Biology. Humana Press, Totowa, N.J., pp 365-386).

Other factors that can influence PCR primer design include primer length, melting temperature (Tm), and G/C content, specificity, complementary primer sequences, and 3′-end sequence. In general, optimal PCR primers are generally 17-30 bases in length, and contain about 20-80%, such as, for example, about 50-60% G+C bases, and exhibit Tm's between 50 and 80° C., e.g. about 50 to 70° C.

For further guidelines for PCR primer and probe design see, e.g. Dieffenbach, C W. et al, “General Concepts for PCR Primer Design” in: PCR Primer, A Laboratory Manual, Cold Spring Harbor Laboratory Press,. New York, 1995, pp. 133-155; Innis and Gelfand, “Optimization of PCRs” in: PCR Protocols, A Guide to Methods and Applications, CRC Press, London, 1994, pp. 5-11; and Plasterer, T. N. Primerselect: Primer and probe design. Methods MoI. Biol. 70:520-527 (1997), the entire disclosures of which are hereby expressly incorporated by reference.

Table 1 provides the GeneBank accession numbers and Entrez ID numbers for each of the response indicator genes of the invention. Based on these sequences, primers, probes, and amplicon sequences can be determined using methods known in the art.

### MassARRAY® System

In MassARRAY-based methods, such as the exemplary method developed by Sequenom, Inc. (San Diego, Calif.) following the isolation of RNA and reverse transcription, the obtained cDNA is spiked with a synthetic DNA molecule (competitor), which matches the targeted cDNA region in all positions, except a single base, and serves as an internal standard. The cDNA/competitor mixture is PCR amplified and is subjected to a post-PCR shrimp alkaline phosphatase (SAP) enzyme treatment, which results in the dephosphorylation of the remaining nucleotides. After inactivation of the alkaline phosphatase, the PCR products from the competitor and cDNA are subjected to primer extension, which generates distinct mass signals for the competitor- and cDNA-derived PCR products. After purification, these products are dispensed on a chip array, which is pre-loaded with components needed for analysis with matrix-assisted laser desorption ionization time-of-flight mass spectrometry (MALDI-TOF MS) analysis. The cDNA present in the reaction is then quantified by analyzing the ratios of the peak areas in the mass spectrum generated. For further details see, e.g. Ding and Cantor, Proc. Natl. Acad. Sci. USA 100:3059-3064 (2003).

### Other PCR-Based Methods

Further PCR-based techniques that can find use in the methods disclosed herein include, for example, BeadArray® technology (Illumina, San Diego, Calif.; Oliphant et al., Discovery of Markers for Disease (Supplement to Biotechniques), June 2002; Ferguson et al., Analytical Chemistry 72:5618 (2000)); BeadsArray for Detection of Gene Expression® (BADGE), using the commercially available LuminexlOO LabMAP® system and multiple color-coded microspheres (Luminex Corp., Austin, Tex.) in a rapid assay for gene expression (Yang et al., Genome Res. 11:1888-1898 (2001)); and high coverage expression profiling (HiCEP) analysis (Fukumura et al., Nucl. Acids. Res. 31(16) e94 (2003).

### Microarrays

Expression levels of a gene of interest can also be assessed using the microarray technique. In this method, polynucleotide sequences of interest (including cDNAs and oligonucleotides) are arrayed on a substrate. The arrayed sequences are then contacted under conditions suitable for specific hybridization with detectably labeled cDNA generated from mRNA of a sample. As in the RT-PCR method, the source of mRNA typically is total RNA isolated from a tumor sample, and optionally from normal tissue of the same patient as an internal control or cell lines. mRNA can be extracted, for example, from frozen or archived paraffin-embedded and fixed (e.g. formalin-fixed) tissue samples.

For example, PCR amplified inserts of cDNA clones of a gene to be assayed are applied to a substrate in a dense array. Usually at least 10,000 nucleotide sequences are applied to the substrate. For example, the microarrayed genes, immobilized on the microchip at 10,000 elements each, are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After washing under stringent conditions to remove non-specifically bound probes, the chip is scanned by confocal laser microscopy or by another detection method, such as a CCD camera. Quantitation of hybridization of each arrayed element allows for assessment of corresponding mRNA abundance.

With dual color fluorescence, separately labeled cDNA probes generated from two sources of RNA are hybridized pair wise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et at, Proc. Natl. Acad. Sci. USA 93(2):106-149 (1996)). Microarray analysis can be performed on commercially available equipment, following the manufacturer's protocols, such as by using the Affymetrix GenChip® technology, or Incyte's microarray technology.

### Serial Analysis of Gene Expression (SAGE)

Serial analysis of gene expression (SAGE) is a method that allows the simultaneous and quantitative analysis of a large number of gene transcripts, without the need of providing an individual hybridization probe for each transcript. First, a short sequence tag (about 10-14 bp) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag. For more details see, e.g. Velculescu et al., Science 270:484-487 (1995); and Velculescu et al., Cell 88:243-51 (1997).

### Gene Expression Analysis by Nucleic Acid Sequencing

Nucleic acid sequencing technologies are suitable methods for analysis of gene expression. The principle underlying these methods is that the number of times a cDNA sequence is detected in a sample is directly related to the relative expression of the mRNA corresponding to that sequence. These methods are sometimes referred to by the term Digital Gene Expression (DGE) to reflect the discrete numeric property of the resulting data. Early methods applying this principle were Serial Analysis of Gene Expression (SAGE) and Massively Parallel Signature Sequencing (MPSS). See, e.g., S. Brenner, et al., Nature Biotechnology 18(6):630-634 (2000). More recently, the advent of “next-generation” sequencing technologies has made DGE simpler, higher throughput, and more affordable. As a result, more laboratories are able to utilize DGE to screen the expression of more genes in more individual patient samples than previously possible. See, e.g., J. Marioni, Genome Research 18(9):1509-1517 (2008); R. Morin, Genome Research 18(4):610-621 (2008); A. Mortazavi, Nature Methods 5(7):621-628 (2008); N. Cloonan, Nature Methods 5(7):613-619 (2008).

Isolating RNA from Body Fluids

Methods of isolating RNA for expression analysis from blood, plasma and serum (see for example, Tsui NB et al. (2002) Clin. Chem. 48,1647-53 and references cited therein) and from urine (see for example, Boom R et al. (1990) J Clin Microbiol. 28, 495-503 and reference cited therein) have been described.

### Immunohistochemistry

Immunohistochemistry methods are also suitable for detecting the expression levels of genes and applied to the method disclosed herein. Antibodies (e.g., monoclonal antibodies) that specifically bind a gene product of a gene of interest can be used in such methods. The antibodies can be detected by direct labeling of the antibodies themselves, for example, with radioactive labels, fluorescent labels, hapten labels such as biotin, or an enzyme such as horse radish peroxidase or alkaline phosphatase. Alternatively, unlabeled primary antibody can be used in conjunction with a labeled secondary antibody specific for the primary antibody. Immunohistochemistry protocols and kits are well known in the art and are commercially available.

### Proteomics

The term “proteome” is defined as the totality of the proteins present in a sample (e.g. tissue, organism, or cell culture) at a certain point of time. Proteomics includes, among other things, study of the global changes of protein expression in a sample (also referred to as “expression proteomics”). Proteomics typically includes the following steps: (1) separation of individual proteins in a sample by 2-D gel electrophoresis (2-D PAGE); (2) identification of the individual proteins recovered from the gel, e.g. my mass spectrometry or N-terminal sequencing, and (3) analysis of the data using bioinformatics.

General Description of the mRNA Isolation, Purification and Amplification

The steps of a representative protocol for profiling gene expression using fixed, paraffin-embedded tissues as the RNA source, including mRNA isolation, purification, primer extension and amplification are provided in various published journal articles. (See, e.g., T. E. Godfrey et al,. J. Molec. Diagnostics 2: 84-91 (2000); K. Specht et al., Am. J. Pathol. 158: 419-29 (2001), M. Cronin, et al., Am J Pathol 164:35-42 (2004)). Briefly, a representative process starts with cutting a tissue sample section (e.g. about 10 μm thick sections of a paraffin-embedded tumor tissue sample). The RNA is then extracted, and protein and DNA are removed. After analysis of the RNA concentration, RNA repair is performed if desired. The sample can then be subjected to analysis, e.g., by reverse transcription using gene specific promoters followed by PCR.

### Coexpression Analysis

The present invention provides genes that co-express with particular response indicator genes that have been identified as having a correlation with sensitivity or resistance to a PI3K inhibitor. To perform particular biological processes, genes often work together in a concerted way, i.e. they are co-expressed. Co-expressed gene groups identified for a disease process like cancer can also serve as response indicator genes. Such co-expressed genes can be assayed in lieu of, or in addition to, assaying of the response indicator gene with which they co-express.

One skilled in the art will recognize that many co-expression analysis methods now known or later developed will fall within the scope and spirit of the present invention. These methods may incorporate, for example, correlation coefficients, co-expression network analysis, clique analysis, etc., and may be based on expression data from RT-PCR, microarrays, sequencing, and other similar technologies. For example, gene expression clusters can be identified using pair-wise analysis of correlation based on Pearson or Spearman correlation coefficients. (See e.g, Pearson K. and Lee A., Biometrika 2:357 (1902); C. Spearman, Amer. J. Psychol. 15:72-101 (1904); J. Myers, A. Well, Research Design and Statistical Analysis, p. 508 (2^{th} Ed., 2003).) In general, a correlation coefficient of equal to or greater than 0.3 is considered to be statistically significant in a sample size of at least 20. (See e.g., G. Norman, D. Streiner, Biostatistics: The Bare Essentials, 137-138 (3^{rd} Ed. 2007).)

### Reference Normalization

In order to minimize expression measurement variations due to non-biological variations in samples, e.g., the amount and quality of expression product to be measured, raw expression level data measured for a gene product (e.g., cycle threshold (Ct) measurements obtained by qRT-PCR) may be normalized relative to the mean expression level data obtained for one or more reference genes. Examples of reference genes include housekeeping genes, such as GAPDH. Alternatively, all of the assayed genes or a large subset thereof may also concurrently serve as reference genes and normalization can be based on the mean or median signal (Ct) of all of the assayed genes or a subset thereof (often referred to as “global normalization” approach). On a gene-by-gene basis, measured normalized amount of a patient tumor mRNA may be compared to the amount found in a cancer tissue reference set. See e.g., Cronin, M. et al., Am. Soc. Investigative Pathology 164:35-42 (2004). The normalization may be carried out such that a one unit increase in normalized expression level of a gene product generally reflects a 2-fold increase in quantity of expression product present in the sample. For further information on normalization techniques applicable to qRT-PCR data from tumor tissue, see e.g., Silva, S. et al. (2006) BMC Cancer 6, 200; deKok, J. et al. (2005) Laboratory Investigation 85, 154-159.

### Kits of the Invention

The materials for use in the methods of the present invention are suited for preparation of kits produced in accordance with well known procedures. The present invention thus provides kits comprising agents, which may include gene-specific or gene-selective probes and/or primers, for quantitating the expression of the disclosed genes. Such kits may optionally contain reagents for the extraction of RNA from tumor samples, in particular, fixed paraffin-embedded tissue samples and/or reagents for RNA amplification. In addition, the kits may optionally comprise the reagent(s) with an identifying description or label or instructions relating to their use in the methods of the present invention. The kits may comprise containers (including microliter plates suitable for use in an automated implementation of the method), each with one or more of the various reagents (typically in concentrated form) utilized in the methods, including, for example, pre-fabricated microarrays, buffers, the appropriate nucleotide triphosphates (e.g., dATP, dCTP, dGTP and dTTP; or rATP, rCTP, rGTP and UTP), reverse transcriptase, DNA polymerase, RNA polymerase, and one or more probes and primers of the present invention (e.g., appropriate length poly(T) or random primers linked to a promoter reactive with the RNA polymerase). Mathematical algorithms used to estimate or quantify prognostic or predictive information are also properly potential components of kits.

### Reports

The methods of this invention are suited for the preparation of reports summarizing the predictions resulting from the methods of the present invention. A “report” as described herein, is an electronic or tangible document that includes elements that provide information of interest relating to a likelihood assessment and its results. A subject report includes at least a likelihood assessment, e.g., an indication as to the likelihood that a cancer patient will be sensitive to a treatment comprising a PI3K inhibitor. A subject report can be completely or partially electronically generated, e.g., presented on an electronic display (e.g., computer monitor). A report can further include one or more of: 1) information regarding the testing facility; 2) service provider information; 3) patient data; 4) sample data; 5) an interpretive report, which can include various information including: a) indication; b) test data, where test data can include a normalized level of one or more genes of interest, and 6) other features.

The present invention therefore provides methods of creating reports and the reports resulting therefrom. The report may include a summary of the expression levels of the RNA transcripts, or the expression products of such RNA transcripts, for certain genes in the cells obtained from the patient's tumor tissue. The report may include a prediction that the patient has an increased likelihood of sensitivity or resistance to treatment with PI3K inhibitor. The report may include a recommendation for a treatment modality such as surgery alone or surgery in combination with a treatment comprising a PI3K inhibitor. The report may be presented in electronic format or on paper.

Thus, in some embodiments, the methods of the present invention further include generating a report that includes information regarding the patient's likelihood of sensitivity or resistance to a PI3K inhibitor, such as GDC-0941. For example, the methods of the present invention can further include a step of generating or outputting a report providing the results of a patient response likelihood assessment, which can be provided in the form of an electronic medium (e.g., an electronic display on a computer monitor), or in the form of a tangible medium (e.g., a report printed on paper or other tangible medium).

A report that includes information regarding the likelihood that a patient will exhibit sensitivity to treatment with a PI3K inhibitor, such as GDC-0941, is provided to a user. An assessment as to the likelihood that a cancer patient will be sensitive to treatment with a PI3K inhibitor, such as GDC-0941, is referred to as a “response likelihood assessment” or “likelihood assessment.” A person or entity who prepares a report (“report generator”) may also perform the likelihood assessment. The report generator may perform one or more of: a) sample gathering; b) sample processing; c) measuring a level of a response indicator gene expression product(s); d) measuring a level of a reference gene product(s); and e) determining a normalized level of a response indicator gene expression product(s). Alternatively, an entity other than the report generator can perform one or more sample gathering, sample processing, and data generation.

The term “user” or “client” refers to a person or entity to whom a report is transmitted, and may be the same person or entity who does one or more of the following: a) collects a sample; b) processes a sample; c) provides a sample or a processed sample; and d) generates data (e.g., level of a predictive gene expression product(s); level of a reference gene product(s); normalized level of a predictive gene expression product(s)) for use in the likelihood assessment. In some cases, the person or entity who provides sample collection and/or sample processing and/or data generation, and the person who receives the results and/or report may be different persons, but are both referred to as “users” or “clients.” In certain embodiments, e.g., where the methods are completely executed on a single computer, the user or client provides for data input and review of data output. A “user” can be a health professional (e.g., a clinician, a laboratory technician, a physician (e.g., an oncologist, surgeon, pathologist), etc.).

In embodiments where the user only executes a portion of the method, the individual who, after computerized data processing according to the methods of the invention, reviews data output (e.g., results prior to release to provide a complete report, a complete, or reviews an “incomplete” report and provides for manual intervention and completion of an interpretive report) is referred to herein as a “reviewer.” The reviewer may be located at a location remote to the user (e.g., at a service provided separate from a healthcare facility where a user may be located).

Where government regulations or other restrictions apply (e.g., requirements by health, malpractice, or liability insurance), all results, whether generated wholly or partially electronically, are subjected to a quality control routine prior to release to the user.

### Computer-Based Systems and Methods

The methods and systems described herein can be implemented in numerous ways. In one embodiment of the invention, the methods involve use of a communications infrastructure, for example, the internet. Several embodiments of the invention are discussed below. The present invention may also be implemented in various forms of hardware, software, firmware, processors, or a combination thereof. The methods and systems described herein can be implemented as a combination of hardware and software. The software can be implemented as an application program tangibly embodied on a program storage device, or different portions of the software implemented in the user's computing environment (e.g., as an applet) and on the reviewer's computing environment, where the reviewer may be located at a remote site (e.g., at a service provider's facility).

In an embodiment of the invention, during or after data input by the user, portions of the data processing can be performed in the user-side computing environment. For example, the user-side computing environment can be programmed to provide for defined test codes to denote a likelihood “score,” where the score is transmitted as processed or partially processed responses to the reviewer's computing environment in the form of test code for subsequent execution of one or more algorithms to provide a result and/or generate a report in the reviewer's computing environment. The score can be a numerical score (representative of a numerical value) or a non-numerical score representative of a numerical value or range of numerical values (e.g., “A”: representative of a 90-95% likelihood of sensitivity to a PI3K inhibitor; “High”: representative of a greater than 50% chance of sensitivity to a PI3K inhibitor (or some other selected threshold of likelihood); “Low”: representative of a less than 50% chance of sensitivity to a PI3K inhibitor (or some other selected threshold of likelihood), and the like.

As a computer system, the system generally includes a processor unit. The processor unit operates to receive information, which can include test data (e.g., level of a predictive gene product(s); level of a reference gene product(s); normalized level of a predictive gene product(s); and may also include other data such as patient data. This information received can be stored at least temporarily in a database, and data analyzed to generate a report as described above.

Part or all of the input and output data can also be sent electronically. Certain output data (e.g., reports) can be sent electronically or telephonically (e.g., by facsimile, using devices such as fax back). Exemplary output receiving devices can include a display element, a printer, a facsimile device and the like. Electronic forms of transmission and/or display can include email, interactive television, and the like. In an embodiment of the invention, all or a portion of the input data and/or output data (e.g., usually at least the final report) are maintained on a web server for access, preferably confidential access, with typical browsers. The data may be accessed or sent to health professionals as desired. The input and output data, including all or a portion of the final report, can be used to populate a patient's medical record that may exist in a confidential database as the healthcare facility.

The present invention also contemplates a computer-readable storage medium (e.g., CD-ROM, memory key, flash memory card, diskette, etc.) having stored thereon a program which, when executed in a computing environment, provides for implementation of algorithms to carry out all or a portion of the results of a response likelihood assessment as described herein. Where the computer-readable medium contains a complete program for carrying out the methods described herein, the program includes program instructions for collecting, analyzing and generating output, and generally includes computer readable code devices for interacting with a user as described herein, processing that data in conjunction with analytical information, and generating unique printed or electronic media for that user.

Where the storage medium includes a program that provides for implementation of a portion of the methods described herein (e.g., the user-side aspect of the methods (e.g., data input, report receipt capabilities, etc.)), the program provides for transmission of data input by the user (e.g., via the internet, via an intranet, etc.) to a computing environment at a remote site. Processing or completion of processing of the data is carried out at the remote site to generate a report. After review of the report, and completion of any needed manual intervention, to provide a complete report, the complete report is then transmitted back to the user as an electronic document or printed document (e.g., fax or mailed paper report). The storage medium containing a program according to the invention can be packaged with instructions (e.g., for program installation, use, etc.) recorded on a suitable substrate or a web address where such instructions may be obtained. The computer-readable storage medium can also be provided in combination with one or more reagents for carrying out a response likelihood assessment (e.g., primers, probes, arrays, or such other kit components).

Having described the invention, the same will be more readily understood through reference to the following Examples, which are provided by way of illustration, and are not intended to limit the invention in any way. All citations through the disclosure are hereby expressly incorporated by reference.

### EXAMPLES

### Example 1

The NCI-60 tumor cell line collection is a panel of 60 diverse human cancer cell lines. These highly characterized cell lines were profiled for their in vitro sensitivity to the PI3K inhibitor GDC-0941. Then, genes predictive of the sensitivity or resistance to GDC-0941 were identified.

Cell Lines and Drug Screening

Cell lines used in this study were obtained from the National Cancer Institute Developmental Therapeutics program (NCI DTP) and were maintained in RPMI 1640 media (Invitrogen, Grand Island, N.Y.) containing 10% Fetal Bovine Serum (ATCC, Manassas, Va.). Cell lines were used within six months of receipt from the NCI DTP, abrogating the need for reauthentication. GDC-0941 and NVP-BEZ235 were obtained from Selleck Chemicals (Houston, Tex.) and dissolved in DMSO prior to cell treatments.

In vitro drug sensitivity testing of the NCI-60 tumor cell line collection against GDC-0941 was performed, with two biological replicates, by the National Cancer Institute's Developmental Therapeutics Program as previously described (Skehan et al., J. Natl. Cancer Inst. 82:1107-1112, 1990), on Jun. 14, 2010 and Aug. 9, 2010. GI₅₀ was defined as the drug concentration resulting in a 50% reduction in net protein increase (as measured by sulforhodamine B (SRB) staining) as compared to control cells. Following standardization, cell lines with a standardized GI₅₀ (z-score) ≧0.8 were defined as being resistant and cell lines with a z-score ≦−0.8 were defined as being sensitive to GDC-0941.

Microarray Data, Identification of Differentially Expressed Genes, and Satatistical Analysis

The normalized log₂ mRNA expression data for the NCI-60 tumor cell line collection (Liu et al., Mol. Cancer Ther. 9:1080-1091, 2010) was downloaded from the CellMiner™ database (http://discover.nci.nih.gov/cellminer) and imported into GenePattern™ 3.3 for all downstream analyses (http://www.broad.mit.edu/cancer/software/genepattern). Missing values were estimated with a weighted K-nearest neighbors algorithm (KNNimpute), and two-way average linkage hierarchical cluster analysis was performed using a UPGMA algorithm, with the data being displayed relative to the median expression for each gene. To identify genes differentially expressed between GDC-0941 resistant- and sensitive-tumor cell lines, the Comparative Marker Selection Module (Gould et al., Bioinformatics 22:1924-1925, 2006) of GenePattern™ 3.3 was utilized, employing a nonparametric t-test with a P value cutoff of 0.01. All P values were corrected for multiple testing with the Bonferroni correction. The Gene Set Enrichment Analysis (GSEA) (Subramanian et al., Proc. Natl. Acad. Sci. USA 102:15545-15550, 2005) module of GenePattern™ 3.3 was used to determine the extent to which the basal expression profiles were enriched for a priori defined set of genes from biologically coherent pathways. To correct for multiple hypotheses testing, the false discovery rate (FDR) threshold was set at <0.25.

Correlations between drug sensitivity and gene expression, levels of phosphorylation, mutational status, tumor type, or tissue of origin were estimated by Spearman's rank correlation method, and differences between groups were calculated with Student's t test using Prism® 5.0 (GraphPad, La Jolla, Calif.). All tests of significance were two-sided, and P values <0.05 were considered significant.

Results

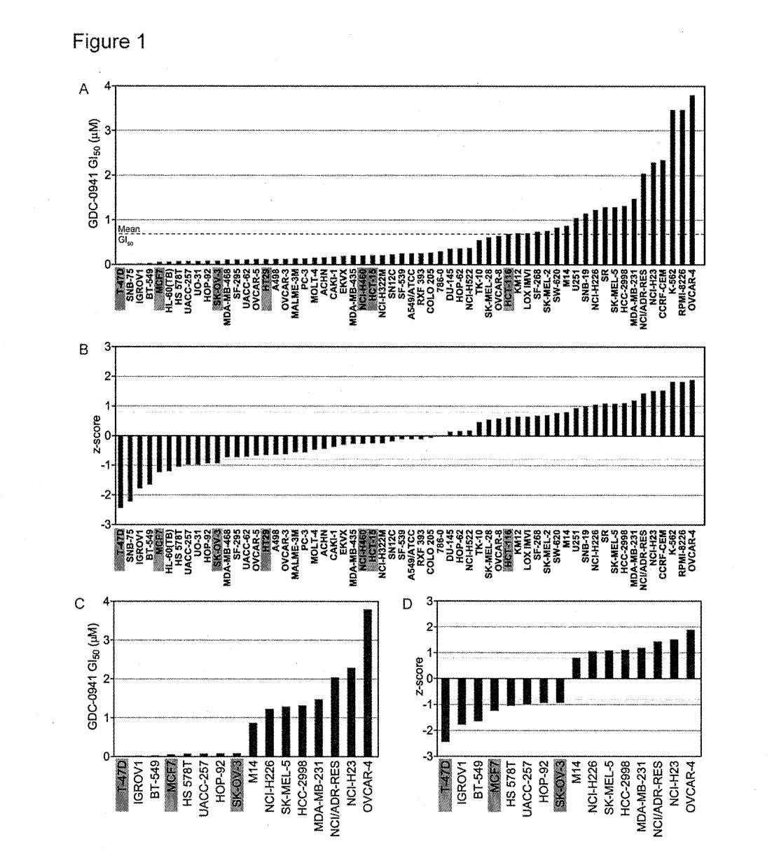

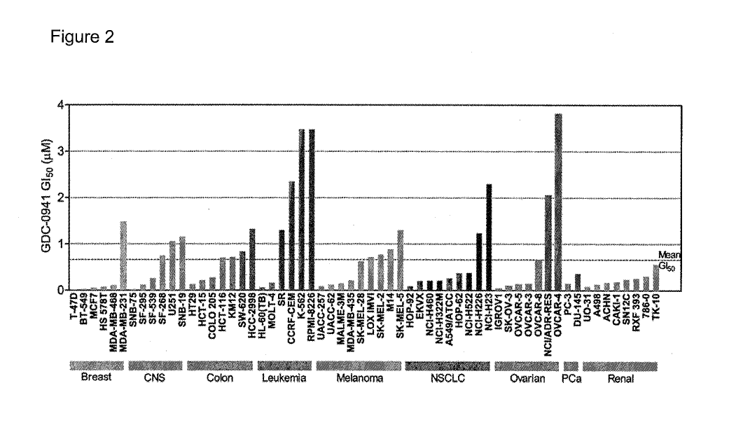

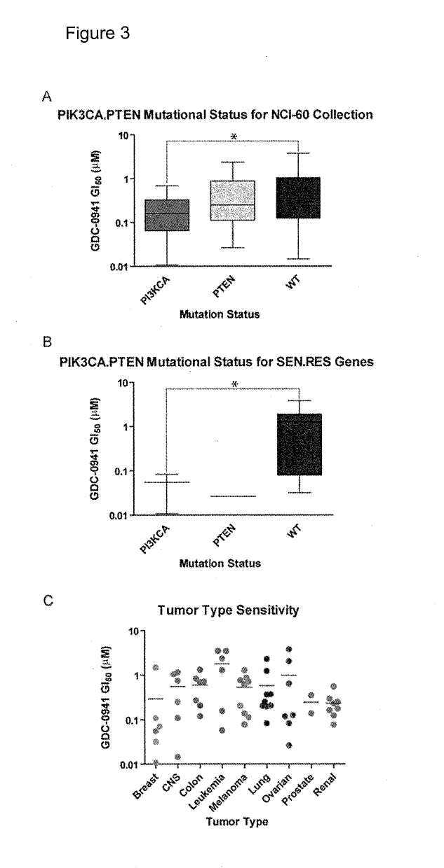

As shown in FIG. 1, the NCI-60 tumor cell lines exhibited over a 2-log range in GI₅₀ values, ranging from 0.011 μM to 3.80 μM (mean=0.66 μM; FIG. 1A; Table 1), with all tumor types displaying a wide range of sensitivities to GDC-0941 (FIG. 2). As previously shown in a panel of 54 breast cancer cell lines (O'Brien et al., Clin. Cancer Res. 16:3670-3683, 2010), an association was found between PI3KCA mutational status amongst cells in the NCI-60 tumor cell line collection and sensitivity to GDC-0941 (P=0.044; FIG. 3), whereas there was no association between PTEN loss and response to GDC-0941 (P=0.31; FIG. 3). Following standardization of all GI₅₀ values, cell lines with standardized GI₅₀ values ≧0.8 SD from the mean GI₅₀ were deemed resistant, and cell lines with standardized GI₅₀ values ≦−0.8 SD from the mean GI₅₀ were deemed sensitive (FIG. 1B).

Profiles of basal mRNA gene expression for the NCI-60 tumor cell line collection were generated using data publicly available (Liu et al., Mol. Cancer Ther. 9:1080-1091, 2010) through the CellMiner™ database (http://discover.nci.nih.gov/cellminer). Only those GDC-0941-sensitive and-resistant tumor cell lines that were epithelial in origin (breast, colon, NSCLC, renal, ovarian, prostate, and melanoma) were analyzed, and tumor cell lines derived from leukemias and tumors of CNS-origin were not further analyzed (FIGS. 1C and 1D). This segregation of the cell lines allowed profiling of a collection of tumor cell lines (T-47D, IGROV1, BT-549, MCF7, HS 578T, UACC-257, HOP-92, SK-OV-3, M14, NCI-H226, SK-MEL-5, HCC-2998, MDA-MB-231, NCI/ADR-RES, NCI-H23, and OVCAR-4) that still represents a heterogeneous population of tumor types while simultaneously avoiding variability in gene expression resulting from inclusion of mesenchymal tumors in the analysis.

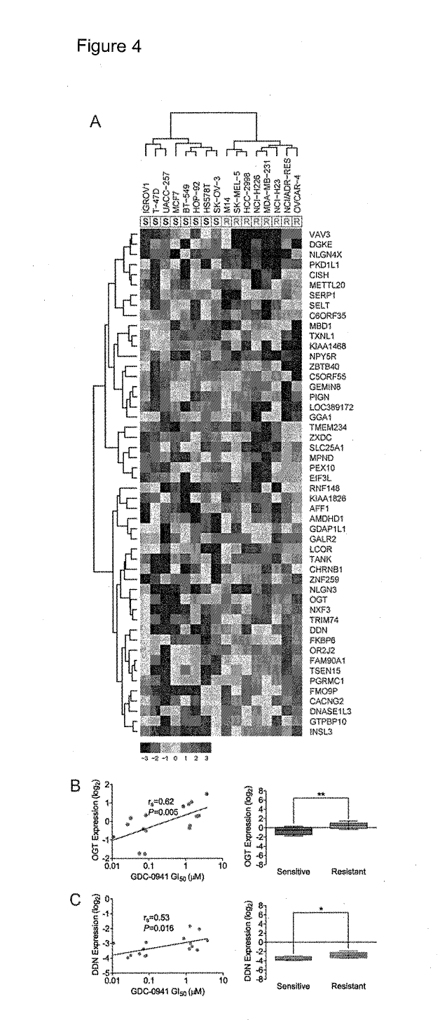

Using the Comparative Marker Selection Module (Gould et al., Bioinformatics 22:1924-1925, 2006) of GenePattern™ 3.3, 221 genes were identified as being differentially expressed between GDC-0941-sensitive and GDC-0941-resistant tumor cell lines with a P value <0.01 (Tables 2 and 3). Unsupervised hierarchical clustering of the top 50 differentially expressed genes (rank order based on P value) correctly classified the 16 analyzed tumor cell lines (T-47D, IGROV1, BT-549, MCF7, HS 578T, UACC-257, HOP-92, SK-OV-3, M14, NCI-H226, SK-MEL-5, HCC-2998, MDA-MB-231, NCI/ADR-RES, NCI-H23, and OVCAR-4) into GDC-0941-sensitive or GDC-0941-resistant subgroups (FIG. 4A). Table 4 lists the top differentially expressed genes in GDC-0941-sensitive and GDC-0941-resistant tumor cell lines, along with associated metrics used in the analysis.

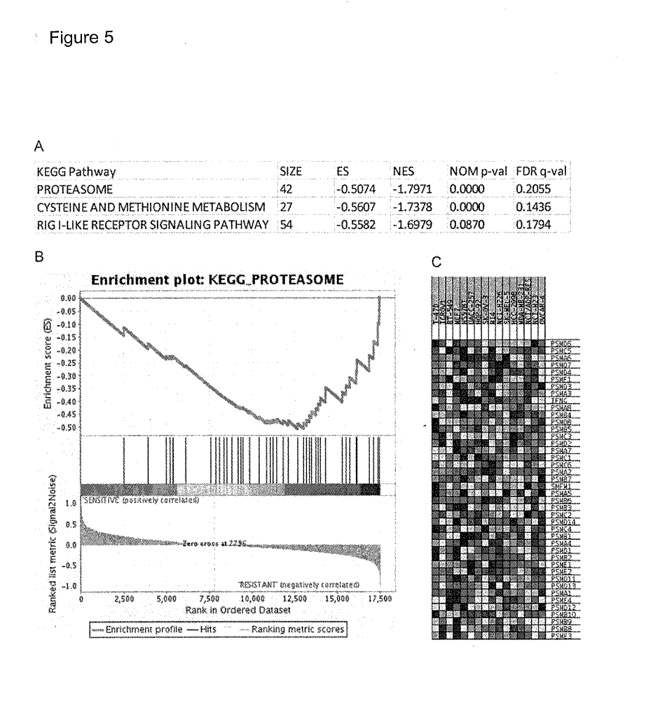

In order to assess the more subtle contributions of coherent cellular pathways in distinguishing GDC-0941-sensitive and GDC-0941-resistant tumor cell lines, Gene Set Enrichment Analysis (Subramanian et al., Proc. Natl. Acad. Sci. USA 102:15545-15550, 2005) was performed. Using an FDR cut-off of <25%, several KEGG pathways were identified that specifically enriched in GDC-0941-resistant tumor cell lines, including the proteasome and cysteine and methionine metabolic pathways (FIG. 5).

TABLE 1

| CELL LINE | GDC-0941 GI50 (μM) |
|---|---|
| T-47D | 0.011 |
| SNB-75 | 0.014 |
| IGROV1 | 0.026 |
| BT-549 | 0.032 |
| MCF7 | 0.055 |
| HL-60(TB) | 0.058 |
| HS 578T | 0.071 |
| UACC-257 | 0.078 |
| UO-31 | 0.078 |
| HOP-92 | 0.083 |
| SK-OV-3 | 0.083 |
| MDA-MB-468 | 0.110 |
| SF-295 | 0.110 |
| UACC-62 | 0.112 |
| OVCAR-5 | 0.117 |
| HT29 | 0.120 |
| A498 | 0.123 |
| OVCAR-3 | 0.126 |
| MALME-3M | 0.138 |
| PC-3 | 0.138 |
| MOLT-4 | 0.155 |
| ACHN | 0.162 |
| CAKI-1 | 0.178 |
| EKVX | 0.195 |
| MDA-MB-435 | 0.204 |
| NCI-H460 | 0.204 |
| HCT-15 | 0.209 |
| NCI-H322M | 0.209 |
| SN12C | 0.229 |
| SF-539 | 0.251 |
| A549/ATCC | 0.251 |
| RXF 393 | 0.251 |
| COLO 205 | 0.269 |
| 786-0 | 0.295 |
| DU-145 | 0.355 |
| HOP-62 | 0.363 |
| NCI-H522 | 0.372 |
| TK-10 | 0.550 |
| SK-MEL-28 | 0.617 |
| OVCAR-8 | 0.646 |
| HCT-116 | 0.692 |
| KM12 | 0.708 |
| LOX IMVI | 0.708 |
| SF-268 | 0.741 |
| SK-MEL-2 | 0.759 |
| SW-620 | 0.832 |
| M14 | 0.871 |
| U251 | 1.047 |
| SNB-19 | 1.148 |
| NCI-H226 | 1.230 |
| SR | 1.288 |
| SK-MEL-5 | 1.288 |
| HCC-2998 | 1.318 |
| MDA-MB-231 | 1.479 |
| NCI/ADR-RES | 2.042 |
| NCI-H23 | 2.291 |
| CCRF-CEM | 2.344 |
| K-562 | 3.467 |
| RPMI-8226 | 3.467 |
| OVCAR-4 | 3.802 |

TABLE 2
mRNA Upregulated in GDC-0941 Sensitive Cell Lines

| GENE SYMBOL | mRNA Accession | DESCRIPTION | T-TEST | P VALUE |
|---|---|---|---|---|
| VAV3 | NM_001079874 | vav 3 oncogene | 3.99349542 | 5.99E−03 |
| DGKE | NM_003647 | diacylglycerol kinase, epsilon 64 kDa | 3.30970125 | 7.98E−03 |
| MBD1 | NM_001204136 | methyl-CpG binding domain protein 1 | 3.177771087 | 5.99E−03 |
| SERP1 | NM_014445 | stress-associated endoplasmic reticulum protein 1 | 2.971906494 | 5.99E−03 |
| TXNL1 | NM_004786 | thioredoxin-like 1 | 2.873839253 | 5.99E−03 |
| NLGN4X | NM_020742 | neuroligin 4, X-linked | 2.870411446 | 5.99E−03 |
| C1ORF91 | NM_019118.3 | chromosome 1 open reading frame 91 | 2.758387909 | 5.99E−03 |
| SLC25A1 | NM_001256534 | solute carrier family 25 (mitochondrial carrier; citrate transporter), | 2.502673578 | 5.99E−03 |
| | | member 1 | | |
| ZBTB40 | NM_001083621 | zinc finger and BTB domain containing 40 | 2.456580697 | 7.98E−03 |
| FAM51A1 | NM_001042479.1 | family with sequence similarity 51, member A1 | 2.359915006 | 5.99E−03 |
| LOC116349 | NM_138464.2 | hypothetical protein BC014011 | 2.330401437 | 7.98E−03 |
| KIAA1468 | NM_020854 | KIAA1468 | 2.265407468 | 5.99E−03 |
| PIGN | NM_012327 | phosphatidylinositol glycan, class N | 2.227730745 | 5.99E−03 |
| PKD1L1 | NM_138295 | polycystic kidney disease 1 like 1 | 2.132700738 | 5.99E−03 |
| SELT | NM_016275 | selenoprotein T | 1.916286004 | 5.99E−03 |
| CISH | NM_013324 | cytokine inducible SH2-containing protein | 1.848360541 | 7.98E−03 |
| MGC50559 | NM_001135863.1 | hypothetical protein MGC50559 | 1.80851638 | 7.98E−03 |
| NPY5R | NM_006174 | neuropeptide Y receptor Y5 | 1.789658401 | 7.98E−03 |
| PEX10 | NM_002617 | peroxisome biogenesis factor 10 | 1.751951298 | 5.99E−03 |
| C6ORF35 | NM_018452.3 | chromosome 6 open reading frame 35 | 1.723099581 | 5.99E−03 |
| ZXDC | NM_001040653 | ZXD family zinc finger C | 1.646747618 | 5.99E−03 |
| LOC389172 | discontinued | hypothetical LOC389172 | 1.641425061 | 5.99E−03 |
| GGA1 | NM_001001560.2 | golgi associated, gamma adaptin ear containing, ARF binding protein 1 | 1.616358413 | 7.98E−03 |
| FLJ14981 | NM_001159846.1 | hypothetical protein FLJ14981 | 1.435041411 | 5.99E−03 |
| EIF3S6IP | NM_001242923.1 | eukaryotic translation initiation factor 3, subunit 6 interacting protein | 1.304194203 | 7.98E−03 |
| ANKRD10 | NM_017664 | ankyrin repeat domain 10 | 1.189784329 | 7.98E−03 |
| DNAJC3 | NM_006260 | DnaJ (Hsp40) homolog, subfamily C, member 3 | 0.945095693 | 5.99E−03 |
| SLC5A9 | NM_001011547 | solute carrier family 5 (sodium/glucose cotransporter), member 9 | 0.841257222 | 5.99E−03 |

TABLE 3
mRNA Upregulated in GDC-0941 Resistant Cell Lines

| GENE SYMBOL | mRNA Accession | DESCRIPTION | T-TEST | P VALUE |
|---|---|---|---|---|
| DJ328E19.C1.1 | NM_015383.1 | hypothetical protein DJ328E19.C1.1 | −0.028085347 | 2.00E−03 |
| STARS | NM_139166.4 | striated muscle activator of Rho-dependent signaling | −0.130229962 | 2.00E−03 |
| SNTG2 | NM_018968 | syntrophin, gamma 2 | −0.214893711 | 2.00E−03 |
| GPR88 | NM_022049 | G-protein coupled receptor 88 | −0.227129067 | 3.99E−03 |
| C8ORF8 | AJ301561.1 | chromosome 8 open reading frame 8 | −0.29883905 | 2.00E−03 |
| UROC1 | NM_001165974 | urocanase domain containing 1 | −0.354294985 | 2.00E−03 |
| POU1F1 | NM_000306 | POU domain, class 1, transcription factor 1 (Pitt, growth hormone factor 1) | −0.448653769 | 9.98E−03 |
| CACNA1E | NM_000721 | calcium channel, voltage-dependent, alpha 1E subunit | −0.496014269 | 7.98E−03 |
| TAPBP | NM_003190 | TAP binding protein (tapasin) | −0.561978408 | 2.00E−03 |
| ASCL3 | NM_020646 | achaete-scute complex (Drosophila) homolog-like 3 | −0.639334292 | 2.00E−03 |
| MGC39900 | NM_194324.2 | hypothetical protein MGC39900 | −0.677404822 | 9.98E−03 |
| FAM71A | NM_153606 | family with sequence similarity 71, member A | −0.72356365 | 2.00E−03 |
| CFC1 | NM_032545 | cripto, FRL-1, cryptic family 1 | −0.740205123 | 9.98E−03 |
| KIAA0892 | NM_015329 | KIAA0892 | −0.761472417 | 2.00E−03 |
| SDS | NM_006843.2 | serine dehydratase | −0.76605754 | 7.98E−03 |
| KIAA0265 | NM_014997 | KIAA0265 protein | −0.792094794 | 3.99E−03 |
| KRTAP9-9 | NM_030975 | keratin associated protein 9-9 | −0.806170016 | 9.98E−03 |
| C1ORF19 | NM_052965 | chromosome 1 open reading frame 19 | −0.811778761 | 2.00E−03 |
| SDR-O | NM_148897 | orphan short-chain dehydrogenase/reductase | −0.841738935 | 2.00E−03 |
| CEACAM8 | NM_001816 | carcinoembryonic antigen-related cell adhesion molecule 8 | −0.899225331 | 2.00E−03 |
| LOC339903 | NM_001205272.1 | hypothetical protein LOC339903 | −0.906845915 | 2.00E−03 |
| LOC441351 | discontinued | hypothetical gene supported by BX537900 | −0.943172922 | 2.00E−03 |
| DCST1 | NM_001143687 | DC-STAMP domain containing 1 | −0.946738063 | 2.00E−03 |
| SLIC1 | NM_182854 | selectin ligand interactor cytoplasmic-1 | −0.965059993 | 2.00E−03 |
| RBPSUHL | NM_014276.2 | recombining binding protein suppressor of hairless (Drosophila)-like | −0.973429189 | 2.00E−03 |
| ARR3 | NM_004312 | arrestin 3, retinal (X-arrestin) | −0.981367343 | 2.00E−03 |
| ANGPT4 | NM_015985 | angiopoietin 4 | −0.984185531 | 9.98E−03 |
| SIGLEC11 | NM_001135163 | sialic acid binding Ig-like lectin 11 | −0.999507759 | 9.98E−03 |
| CASP14 | NM_012114.2 | caspase 14, apoptosis-related cysteine protease | −1.011312123 | 9.98E−03 |
| ODF3 | NM_053280 | outer dense fiber of sperm tails 3 | −1.036910859 | 9.98E−03 |
| TXNDC2 | NM_001098529.1 | thioredoxin domain containing 2 (spermatozoa) | −1.046506966 | 3.99E−03 |
| FCAMR | NM_001122979 | Fc receptor, IgA, IgM, high affinity | −1.054646283 | 3.99E−03 |
| PIM1 | NM_001243186 | pim-1 oncogene | −1.066251643 | 3.99E−03 |
| GPR51 | NM_005458.7 | G protein-coupled receptor 51 | −1.067705154 | 9.98E−03 |
| C21ORF77 | NM_144659.5 | chromosome 21 open reading frame 77 | −1.079641581 | 9.98E−03 |
| ABT1 | NM_013375 | activator of basal transcription 1 | −1.08320238 | 3.99E−03 |
| GGTLA1 | NM_001099781. | gamma-glutamyltransferase-like activity 1 | −1.096524221 | 9.98E−03 |
| C1ORF92 | NM_144702.1 | chromosome 1 open reading frame 92 | −1.104733034 | 3.99E−03 |
| FLJ23342 | NM_024631.2 | hypothetical protein FLJ23342 | −1.135985347 | 9.98E−03 |
| FLJ31222 | NR_027254.1 | FLJ31222 protein | −1.148929445 | 2.00E−03 |
| SDK1 | NM_001079653 | sidekick homolog 1 (chicken) | −1.149798931 | 2.00E−03 |
| ATP2B3 | NM_001001344 | ATPase, Ca++ transporting, plasma membrane 3 | −1.152240373 | 2.00E−03 |
| LOC116143 | NM_001256476.1 | hypothetical protein BC014022 | −1.170544342 | 7.98E−03 |
| WFDC5 | NM_145652 | WAP four-disulfide core domain 5 | −1.179645811 | 2.00E−03 |
| ZNF179 | NM_007148.3 | zinc finger protein 179 | −1.218005094 | 2.00E−03 |
| CHRNA4 | NM_000744 | cholinergic receptor, nicotinic, alpha polypeptide 4 | −1.225491802 | 2.00E−03 |
| KIR2DS4 | NM_012314 | killer cell immunoglobulin-like receptor, two domains, short cytoplasmic | −1.236406396 | 9.98E−03 |
| | | tail, 4 | | |
| C8ORF31 | NM_173687 | chromosome 8 open reading frame 31 | −1.24596662 | 9.98E−03 |
| PRG1 | NR_026881.1 | proteoglycan 1, secretory granule | −1.25034164 | 7.98E−03 |
| MATN2 | NM_002380 | matrilin 2 | −1.272344251 | 9.98E−03 |
| ZW10 | NM_004724 | ZW10 homolog, centromere/kinetochore protein (Drosophila) | −1.275847979 | 2.00E−03 |
| PGLYRP1 | NM_005091 | peptidoglycan recognition protein 1 | −1.280318088 | 2.00E−03 |
| DCUN1D5 | NM_032299 | DCN1, defective in cullin neddylation 1, domain containing 5 (S. cerevisiae) | −1.310691162 | 9.98E−03 |
| SNX24 | NM_014035 | sorting nexing 24 | −1.338242888 | 2.00E−03 |
| GPR43 | NM_005306.2 | G protein-coupled receptor 43 | −1.34464462 | 2.00E−03 |
| C10ORF97 | NM_024948 | chromosome 10 open reading frame 97 | −1.359507592 | 9.98E−03 |
| HBD | NM_000518 | hemoglobin, delta | −1.360886839 | 2.00E−03 |
| MYOZ1 | NM_021245 | myozenin 1 | −1.368703537 | 9.98E−03 |
| RBM7 | NM_016090 | RNA binding motif protein 7 | −1.459858045 | 3.99E−03 |
| TSPAN16 | NM_012466 | tetraspanin 16 | −1.468152792 | 2.00E−03 |
| FLJ23657 | NM_001206981.1 | hypothetical protein FLJ23657 | −1.476619859 | 2.00E−03 |
| PIPPIN | NM_014460.3 | RNA-binding protein pippin | −1.478800365 | 2.00E−03 |
| HAT1 | NM_003642 | histone acetyltransferase 1 | −1.480462844 | 7.98E−03 |
| APBB1IP | NM_019043 | amyloid beta (A4) precursor protein-binding, family B, member 1 interacting | −1.488132122 | 2.00E−03 |
| | | protein | | |
| USP29 | NM_020903 | ubiquitin specific protease 29 | −1.48885424 | 2.00E−03 |
| GPR35 | NM_001195381 | G protein-coupled receptor 35 | −1.492651813 | 2.00E−03 |
| BRUNOL5 | NM_001172673.1 | bruno-like 5, RNA binding protein (Drosophila) | −1.515060364 | 2.00E−03 |
| FKBP5 | NM_001145775 | FK506 binding protein 5 | −1.525192477 | 2.00E−03 |
| IFNA21 | NM_002175 | interferon, alpha 21 | −1.530866408 | 2.00E−03 |
| LST1 | NM_001166538.1 | leukocyte specific transcript 1 | −1.55027096 | 9.98E−03 |
| ABCG4 | NM_001142505 | ATP-binding cassette, sub-family G (WHITE), member 4 | −1.563287153 | 2.00E−03 |
| NCF1 | NM_000265 | neutrophil cytosolic factor 1 (47 kDa, chronic granulomatous disease, | −1.572030724 | 2.00E−03 |
| | | autosomal 1) | | |
| POMT2 | NM_013382 | protein-O-mannosyltransferase 2 | −1.572139834 | 2.00E−03 |
| GIMAP6 | NM_001244071 | GTPase, IMAP family member 6 | −1.575605606 | 9.98E−03 |
| FLJ25369 | NM_152670.2 | hypothetical protein FLJ25369 | −1.576652018 | 9.98E−03 |
| TFAP2E | NM_178548 | transcription factor AP-2 epsilon (activating enhancer binding | −1.580632134 | 2.00E−03 |
| | | protein 2 epsilon) | | |
| IQCA | NM_024726.3 | IQ motif containing with AAA domain | −1.585306001 | 9.98E−03 |
| PRKCG | NM_002739 | protein kinase C, gamma | −1.587359329 | 2.00E−03 |
| FLT3LG | NM_001204502 | fms-related tyrosine kinase 3 ligand | −1.601667253 | 2.00E−03 |
| CRSP6 | NM_004268.4 | cofactor required for Sp1 transcriptional activation, subunit 6, 77 kDa | −1.623205977 | 2.00E−03 |
| KIAA0773 | NM_001031690.2 | KIAA0773 gene product | −1.644451768 | 9.98E−03 |
| DKFZP434M131 | NM_001166137.1 | hypothetical LOC441452 | −1.667890772 | 3.99E−03 |
| GPR45 | NM_007227 | G protein-coupled receptor 45 | −1.674271865 | 9.98E−03 |
| RARS | NM_002887 | arginyl-tRNA synthetase | −1.684971425 | 2.00E−03 |
| KIAA1751 | NM_001080484 | KIAA1751 protein | −1.690678359 | 2.00E−03 |
| P2RY11 | NM_002566 | purinergic receptor P2Y, G-protein coupled, 11 | −1.694533028 | 9.98E−03 |
| CEP164 | NM_014956 | KIAA1052 protein | −1.723207121 | 2.00E−03 |
| C1ORF42 | NM_019060.2 | chromosome 1 open reading frame 42 | −1.731543787 | 9.98E−03 |
| CBL | NM_005188 | Cas-Br—M (murine) ecotropic retroviral transforming sequence | −1.732587607 | 9.98E−03 |
| LMX1A | NM_001174069 | LIM homeobox transcription factor 1, alpha | −1.745879285 | 3.99E−03 |
| PAFAH1B2 | NM_001184746 | platelet-activating factor acetylhydrolase, isoform Ib, beta subunit 30 kDa | −1.746449462 | 2.00E−03 |
| TNFRSF13C | NM_052945 | tumor necrosis factor receptor superfamily, member 13C | −1.756402205 | 2.00E−03 |
| CD8B1 | NM_001178100.1 | CD8 antigen, beta polypeptide 1 (p37) | −1.758458082 | 2.00E−03 |
| MLZE | NM_031415 | melanoma-derived leucine zipper, extra-nuclear factor | −1.763328778 | 2.00E−03 |
| NR2E1 | NM_003269 | nuclear receptor subfamily 2, group E, member 1 | −1.771778691 | 9.98E−03 |
| FETUB | NM_014375 | fetuin B | −1.777581136 | 9.98E−03 |
| KLC3 | NM_177417 | kinesin light chain 3 | −1.779028685 | 9.98E−03 |
| CARD11 | NM_032415 | caspase recruitment domain family, member 11 | −1.797769317 | 2.00E−03 |
| BMP10 | NM_014482 | bone morphogenetic protein 10 | −1.800518081 | 9.98E−03 |
| BRD8 | NM_001164326 | bromodomain containing 8 | −1.80305085 | 2.00E−03 |
| DKFZP686H1423 | NM_144696.4 | hypothetical protein FLJ32940 | −1.808443748 | 2.00E−03 |
| HMCN2 | XM_001726942.2 | hemicentin 2 | −1.816649468 | 2.00E−03 |
| SCN5A | NM_000335 | sodium channel, voltage-gated, type V, alpha (long QT syndrome 3) | −1.842309464 | 2.00E−03 |
| PTS | NM_000317 | 6-pyruvoyltetrahydropterin synthase | −1.85966307 | 9.98E−03 |
| LOC389233 | discontinued | hypothetical LOC389233 | −1.870801629 | 2.00E−03 |
| KLK7 | NM_001207053 | kallikrein 7 (chymotryptic, stratum corneum) | −1.871146908 | 2.00E−03 |
| C1ORF182 | NM_144627 | chromosome 1 open reading frame 182 | −1.882213283 | 2.00E−03 |
| KIAA0999 | NM_025164 | KIAA0999 protein | −1.89446535 | 9.98E−03 |
| LOC387915 | discontinued | hypothetical gene supported by AK000246 | −1.894843242 | 2.00E−03 |
| OTP | NM_032109 | orthopedia homolog (Drosophila) | −1.898780241 | 2.00E−03 |
| HAO1 | NM_017545 | hydroxyacid oxidase (glycolate oxidase) 1 | −1.905087572 | 9.98E−03 |
| KCNJ5 | NM_000890 | potassium inwardly-rectifying channel, subfamily J, member 5 | −1.907634218 | 2.00E−03 |
| FLJ35821 | NM_152589.1 | hypothetical protein FLJ35821 | −1.915732424 | 9.98E−03 |
| CNTN2 | NM_005076 | contactin 2 (axonal) | −1.923191895 | 2.00E−03 |
| NR1H4 | NM_001206977 | nuclear receptor subfamily 1, group H, member 4 | −1.932894899 | 2.00E−03 |
| RASGEF1C | NM_175062 | RasGEF domain family, member 1C | −1.949675606 | 2.00E−03 |
| RAX | NM_013435 | retina and anterior neural fold homeobox | −1.949993478 | 2.00E−03 |
| STYK1 | NM_018423 | serine/threonine/tyrosine kinase 1 | −1.978480025 | 2.00E−03 |
| MGC33367 | NM_144602.2 | hypothetical protein MGC32905 | −1.98551279 | 2.00E−03 |
| HIF3A | NM_022462. | hypoxia inducible factor 3, alpha subunit | −2.019581134 | 2.00E−03 |
| NGB | NM_021257 | neuroglobin | −2.020707127 | 3.99E−03 |
| SH2D4B | NM_207372 | SH2 domain containing 4B | −2.025558639 | 9.98E−03 |
| ACCN4 | NM_018674 | amiloride-sensitive cation channel 4, pituitary | −2.028509102 | 2.00E−03 |
| PRKCQ | NM_001242413 | protein kinase C, theta | −2.032087476 | 2.00E−03 |
| CABP1 | NM_00103367 | calcium binding protein 1 (calbrain) | −2.044690158 | 3.99E−03 |
| ZPBP | NM_001159878 | zona pellucida binding protein | −2.045365993 | 9.98E−03 |
| ESAM | NM_138961 | endothelial cell adhesion molecule | −2.047910673 | 9.98E−03 |
| PIK3R5 | NM_001142633 | phosphoinositide-3-kinase, regulatory subunit 5, p101 | −2.067522638 | 3.99E−03 |
| CDH4 | NM_001252338 | cadherin 4, type 1, R-cadherin (retinal) | −2.07803463 | 9.98E−03 |
| LOC338328 | NM_178172 | high density lipoprotein-binding protein | −2.081311401 | 2.00E−03 |
| SLC32A1 | NM_080552 | solute carrier family 32 (GABA vesicular transporter), member 1 | −2.087332672 | 3.99E−03 |
| KIAA1086 | NM_015174.1 | KIAA1086 | −2.106221336 | 2.00E−03 |
| GNG7 | NM_052847 | guanine nucleotide binding protein (G protein), gamma 7 | −2.148736174 | 9.98E−03 |
| GOT1 | NM_002079 | glutamic-oxaloacetic transaminase 1, soluble (aspartate aminotransferase 1) | −2.151659027 | 2.00E−03 |
| FSHR | NM_000145 | follicle stimulating hormone receptor | −2.154372896 | 2.00E−03 |
| IFNGR2 | NM_005534 | interferon gamma receptor 2 (interferon gamma transducer 1) | −2.159568967 | 2.00E−03 |
| S100A7 | NM_002963 | S100 calcium binding protein A7 (psoriasin 1) | −2.179364634 | 2.00E−03 |
| SFRS15 | NM_001145444.1 | splicing factor, arginine/serine-rich 15 | −2.179601811 | 2.00E−03 |
| SEC14L4 | NM_001161368 | SEC14-like 4 (S. cerevisiae) | −2.182131477 | 2.00E−03 |
| CIDEA | NM_001279 | cell death-inducing DFFA-like effector a | −2.187306206 | 2.00E−03 |
| MYH2 | NM_001100112 | myosin, heavy polypeptide 2, skeletal muscle, adult | −2.209760237 | 2.00E−03 |
| KRTAP12-1 | NM_181686 | keratin associated protein 12-1 | −2.210204462 | 2.00E−03 |
| TBX10 | NM_005995 | T-box 10 | −2.228248868 | 2.00E−03 |
| CYP2U1 | NM_183075 | cytochrome P450, family 2, subfamily U, polypeptide 1 | −2.245998218 | 2.00E−03 |
| ADAM7 | NM_003817 | a disintegrin and metalloproteinase domain 7 | −2.265773592 | 2.00E−03 |
| DKFZP547C195 | NM_001077239.1 | hypothetical protein DKFZp547C195 | −2.278993465 | 9.98E−03 |
| GRP | NM_001012512 | gastrin-releasing peptide | −2.369265694 | 9.98E−03 |
| MGC14151 | NM_032356.3 | hypothetical protein MGC14151 | −2.381954432 | 3.99E−03 |
| NRGN | NM_001126181 | neurogranin (protein kinase C substrate, RC3) | −2.385402678 | 2.00E−03 |
| IGLV6-57 | AF267874 | immunoglobulin lambda variable 6-57 | −2.404237335 | 2.00E−03 |
| RASGRP4 | NM_001146202 | RAS guanyl releasing protein 4 | −2.419862531 | 2.00E−03 |
| C2ORF26 | NM_023016.3 | chromosome 2 open reading frame 26 | −2.421391873 | 3.99E−03 |
| NALP5 | NM_153447.4 | NACHT, leucine rich repeat and PYD containing 5 | −2.424406729 | 9.98E−03 |
| C1ORF177 | NM_001110533 | chromosome 1 open reading frame 177 | −2.446849531 | 9.98E−03 |
| CEP192 | NM_032142 | centrosomal protein 192 kDa | −2.467979769 | 2.00E−03 |
| PDE6B | NM_000283 | phosphodiesterase 6B, cGMP-specific, rod, beta (congenital | −2.494338405 | 3.99E−03 |
| | | stationary night blindness 3, autosomal dominant) | | |
| ZNF502 | NM_001134440 | zinc finger protein 502 | −2.494341878 | 9.98E−03 |
| NMES1 | NM_032413.2 | normal mucosa of esophagus specific 1 | −2.52196283 | 9.98E−03 |
| ATG9B | NM_173681 | ATG9 autophagy related 9 homolog B (S. cerevisiae) | −2.52579614 | 2.00E−03 |
| EBF2 | NM_022659 | early B-cell factor 2 | −2.54771269 | 2.00E−03 |
| CYP17A1 | NM_000102 | cytochrome P450, family 17, subfamily A, polypeptide 1 | −2.561620853 | 3.99E−03 |
| LOC339778 | NM_001105519.1 | hypothetical protein LOC339778 | −2.582862556 | 2.00E−03 |
| DIAPH1 | NM_001079812 | diaphanous homolog 1 (Drosophila) | −2.600656506 | 2.00E−03 |
| RNF183 | NM_145051 | ring finger protein 183 | −2.612879128 | 2.00E−03 |
| FLJ13941 | NM_024848.1 | hypothetical protein FLJ13941 | −2.633839893 | 2.00E−03 |
| HSPBAP1 | NM_024610.5 | HSPB (heat shock 27 kDa) associated protein 1 | −2.639239873 | 2.00E−03 |
| WDR44 | NM_001184965 | WD repeat domain 44 | −2.661414837 | 9.98E−03 |
| UBE2B | NM_003337 | ubiquitin-conjugating enzyme E2B (RAD6 homolog) | −2.66377629 | 2.00E−03 |
| RNF148 | NM_198085 | ring finger protein 148 | −2.672486607 | 9.98E−03 |
| KIAA1826 | NM_032424 | KIAA1826 protein | −2.741348506 | 9.98E−03 |
| OR2J2 | NM_030905 | olfactory receptor, family 2, subfamily J, member 2 | −2.834873529 | 9.98E−03 |
| MLLT2 | NM_001166693.1 | myeloid/lymphoid or mixed-lineage leukemia (trithorax homolog, | −2.851807123 | 2.00E−03 |
| | | Drosophila); translocated to, 2 | | |
| MLR2 | NM_001170765.1 | ligand-dependent corepressor | −2.863788235 | 2.00E−03 |
| LOC116123 | NR_002925.2 | hypothetical protein BC014341 | −2.889906795 | 2.00E−03 |
| TANK | NM_001199135 | TRAF family member-associated NFKB activator | −2.896267736 | 2.00E−03 |
| MGC35366 | NM_152435.2 | hypothetical protein MGC35366 | −2.898360777 | 2.00E−03 |
| NXF3 | NM_022052 | nuclear RNA export factor 3 | −2.910047922 | 9.98E−03 |
| DDN | NM_015086 | dendrin | −2.927580728 | 2.00E−03 |
| ZNF259 | NM_003904 | zinc finger protein 259 | −2.956333574 | 9.98E−03 |
| NLGN3 | NM_001166660 | neuroligin 3 | −2.985516911 | 2.00E−03 |
| CHRNB1 | NM_000747 | cholinergic receptor, nicotinic, beta polypeptide 1 (muscle) | −3.009794517 | 2.00E−03 |
| GDAP1L1 | NM_024034 | ganglioside-induced differentiation-associated protein 1-like 1 | −3.041597793 | 2.00E−03 |
| FKBP6 | NM_001135211 | FK506 binding protein 6, 36 kDa | −3.108070578 | 2.00E−03 |
| OGT | NM_181672 | O-linked N-acetylglucosamine (GlcNAc) transferase (UDP-N- | −3.17565653 | 9.98E−03 |
| | | acetylglucosamine: polypeptide-N-acetylglucosaminyl transferase) | | |
| FAM90A1 | NM_018088 | family with sequence similarity 90, member A1 | −3.209269756 | 2.00E−03 |
| CACNG2 | NM_006078 | calcium channel, voltage-dependent, gamma subunit 2 | −3.214356869 | 2.00E−03 |
| DNASE1L3 | NM_001256560 | deoxyribonuclease I-like 3 | −3.334301804 | 2.00E−03 |
| PGRMC1 | NM_006667 | progesterone receptor membrane component 1 | −3.429693826 | 7.98E−03 |
| GTPBP10 | NM_001042717 | GTP-binding protein 10 | −4.29551105 | 2.00E−03 |
| LOC389633 | NR_036463.1 | similar to FLJ10408 protein | −4.424893582 | 2.00E−03 |
| INSL3 | NM_005543 | insulin-like 3 (Leydig cell) | −5.365202642 | 9.98E−03 |
| GALR2 | NM_003857 | galanin receptor 2 | −5.442236149 | 2.00E−03 |
| TRIM50C | NM_198853.1 | tripartite motif-containing 50C | −5.80636134 | 2.00E−03 |

TABLE 4

| | | | | |
|---|---|---|---|---|
| mRNA Accession | GeneSymbol | Gene Name | T-Test | P-Value |

mRNA Upregulated in GDC-0941 Sensitive Cell Lines

| | | | | |
|---|---|---|---|---|
| NM_001079874 | VAV3 | vav 3 oncogene | 3.99 | 5.99E−03 |
| NM_003647 | DGKE | diacylglycerol kinase, epsilon 64 kDa | 3.31 | 7.98E−03 |
| NM_001204136 | MBD1 | methyl-CpG binding domain protein 1 | 3.18 | 5.99E−03 |
| NM_014445.3 | SERP1 | stress-associated endoplasmic reticulum protein 1 | 2.97 | 5.99E−03 |
| NM_004786 | TXNL1 | thioredoxin-like 1 | 2.87 | 5.99E−03 |
| NM_020742 | NLGN4X | neuroligin 4, X-linked | 2.87 | 5.99E−03 |
| NM_019118 | C1ORF91 | chromosome 1 open reading frame 91 | 2.76 | 5.99E−03 |
| NM_001256534 | SLC25A1 | solute carrier family 25 (mitochondrial carrier; citrate | 2.50 | 5.99E−03 |
| | | transporter), member 1 | | |
| NM_001083621 | ZBTB40 | zinc finger and BTB domain containing 40 | 2.46 | 7.98E−03 |
| NM_017856.2 | FAM51A1 | family with sequence similarity 51, member A1 | 2.36 | 5.99E−03 |
| NM_138464.2 | LOC116349 | hypothetical protein BC014011 | 2.33 | 7.98E−03 |
| NM_020854 | KIAA1468 | KIAA1468 | 2.27 | 5.99E−03 |
| NM_012327 | PIGN | phosphatidylinositol glycan, class N | 2.23 | 5.99E−03 |
| NM_138295 | PKD1L1 | polycystic kidney disease 1 like 1 | 2.13 | 5.99E−03 |
| NM_016275 | SELT | selenoprotein T | 1.92 | 5.99E−03 |
| NM_013324 | CISH | cytokine inducible SH2-containing protein | 1.85 | 7.98E−03 |
| NM_001135863.1 | MGC50559 | hypothetical protein MGC50559 | 1.81 | 7.98E−03 |
| NM_006174 | NPY5R | neuropeptide Y receptor Y5 | 1.79 | 7.98E−03 |
| NM_002617 | PEX10 | peroxisome biogenesis factor 10 | 1.75 | 5.99E−03 |
| NM_018452.4 | C6ORF35 | chromosome 6 open reading frame 35 | 1.72 | 5.99E−03 |

mRNA Upregulated in GDC-0941 Resistant Cell Lines

| | | | | |
|---|---|---|---|---|
| NM_198853 | TRIM50C | tripartite motif-containing 50C | −5.81 | 2.00E−03 |
| NM_003857 | GALR2 | galanin receptor 2 | −5.44 | 2.00E−03 |
| NM_005543 | INSL3 | insulin-like 3 (Leydig cell) | −5.37 | 9.98E−03 |
| NM_001127394 | LOC389633 | similar to FLJ10408 protein | −4.42 | 2.00E−03 |
| NM_001042717 | GTPBP10 | GTP-binding protein 10 | −4.30 | 2.00E−03 |
| NM_006667 | PGRMC1 | progesterone receptor membrane component 1 | −3.43 | 7.98E−03 |
| NM_001256560 | DNASE1L3 | deoxyribonuclease I-like 3 | −3.33 | 2.00E−03 |
| NM_006078 | CACNG2 | calcium channel, voltage-dependent, gamma | −3.21 | 2.00E−03 |
| NM_018088 | FAM90A1 | family with sequence similarity 90, member A1 | −3.21 | 2.00E−03 |
| NM_181672 | OGT | O-linked N-acetylglucosamine (GIcNAc) transferase | −3.18 | 9.98E−03 |
| | | (UDP-N-acetylglucosamine: polypeptide-N- | | |
| | | acetylglucosaminyl transferase) | | |
| NM_001135211 | FKBP6 | FK506 binding protein 6, 36 kDa | −3.11 | 2.00E−03 |
| NM_001256737.1 | GDAP1L1 | ganglioside-induced differentiation-associated | −3.04 | 2.00E−03 |
| NM_000747 | CHRNB1 | cholinergic receptor, nicotinic, beta polypeptide 1 | −3.01 | 2.00E−03 |
| NM_001166660 | NLGN3 | neuroligin 3 | −2.99 | 2.00E−03 |
| NM_003904 | ZNF259 | zinc finger protein 259 | −2.96 | 9.98E−03 |
| NM_015086 | DDN | dendrin | −2.93 | 2.00E−03 |
| NM_022052 | NXF3 | nuclear RNA export factor 3 | −2.91 | 9.98E−03 |
| NM_152435.2 | MGC35366 | hypothetical protein MGC35366 | −2.90 | 2.00E−03 |
| NM_001199135 | TANK | TRAF family member-associated NFKB activator | −2.90 | 2.00E−03 |
| NR_002925 | LOC116123 | hypothetical protein BC014341 | −2.89 | 2.00E−03 |

### Example 2

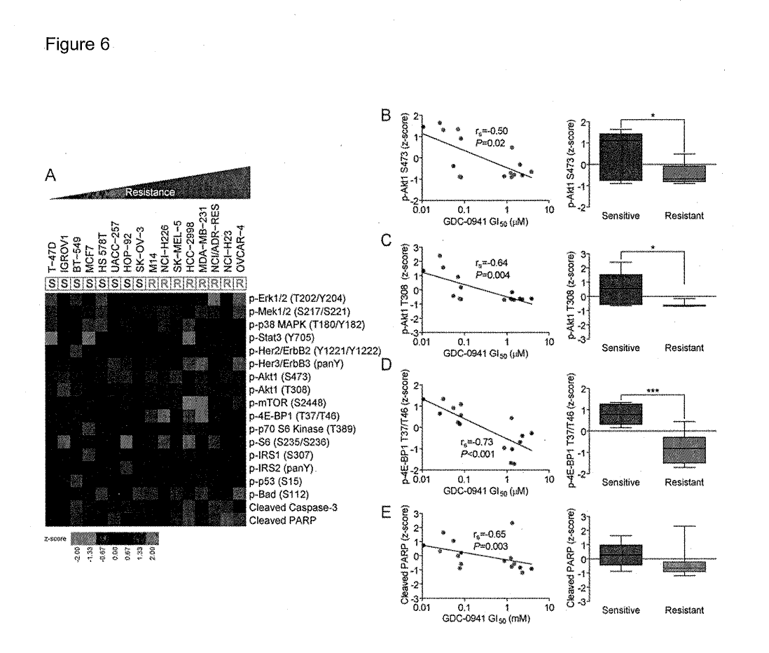

The baseline phosphorylation levels of multiple downstream effectors of the PI3K pathway were next examined, including Akt1 (Ser473), Akt1 (T308), mTOR (S2448), 4E-BP1 (T37/T46), p70 S6 Kinase (T389), and S6 (S235/S236). In addition, the activation status of the MAP kinases, Erk½ (T202/Y204), Mek½ (S217/S221), and p38 MAPK (T180/Y182); transcriptional activators, Stat3 (Tyr705); growth factor and nutrient signaling, Erbb2 (Y1221/Y1222), Erbb3 (panY), IRS1 (S307), IRS2 (panY); and apoptosis signaling molecules, p53 (S 15), Bad (S 112), cleaved caspase-3, and cleaved PARP (FIG. 6A) were assayed.

Phosphorylation and/or cleavage status of Erk½ (T202/Y204), Mek½ (S217/S221), p38 MAPK (T180/Y182), Stat3 (Tyr705), Erbb2 (Y1221/Y1222), Erbb3 (panY), Akt1 (Ser473), Akt1 (T308), mTOR (S2448), 4E-BP1 (T37/T46), p70 S6 Kinase (T389), S6 (S235/S236), IRS1 (S307), IRS2 (panY), p53 (S15), Bad (S112), cleaved caspase-3, cleaved PARP, and Chk2 (T68) were determined using the PathScan® Sandwich ELISA kits (Cell Signaling Technology, Danvers, Mass.) as per manufacturer's instructions. Raw signal intensity (OD₄₅₀) was normalized to total Akt protein levels. For visualization as a heatmap, data was background subtracted, median normalized, and converted into a heatmap using Java TreeView™ (http://jtreeview.sourceforge.net/). Assays were performed in triplicate.

Significantly, levels of phosphorylated Akt1, either at S473 (FIG. 6B; rₛ=−0.50, P=0.02) or T308 (FIG. 6C; rₛ=−0.64, P=0.004), were significantly negatively correlated with resistance to GDC-0941. A significant negative correlation was also observed between the level of phosphorylated 4E-BP1, at T37/T46, and resistance to GDC-0941 (FIG. 6D; rₛ=−0.73, P<0.001). These results suggest that increased baseline activation of several downstream effectors in the PI3K pathway (phosphorylated Akt1 (at S473 and/or T308 and phosphorylated 4E-BP1 at T37/T46) may serve as predictive markers for de novo sensitivity to GDC-0941. Finally, a significant negative correlation was observed between baseline levels of cleaved PARP and resistance to GDC-0941 (FIG. 6E; rₛ=−0.65, P=0.003).

### Example 3

Two differentially expressed genes, O-linked β-N-acetylglucosamine transferase (OGT) and Dendrin (DDN), were further assessed for their roles in mediating sensitivity to inhibition of PI3K by GDC-0941.

SiRNA Knockdown and Cell Viability Assays

ON-TARGETplus™ siRNAs (Thermo Scientific, Waltham, Mass.), containing pools of 4 siRNAs per gene, were utilized for siRNA knockdown experiments as described previously (Harradine et al., Mol. Cancer Res. 9:173-182, 2011). Briefly, 70 μl of cells (1.0×10⁵ cells/ml) were plated in black, clear-bottomed 96-well plates in antibiotic-free RPMI 1640 medium and allowed to adhere overnight. Cells were then transfected with siRNA using DharmaFECT® transfection reagent (Thermo Scientific, Waltham, Mass.) at a final concentration of 25 nM. Following a 4 hour incubation, 10 μl per well of GDC-0941 or NVP-BEZ235 were then added for a total assay volume of 100 μl. Assays were performed in triplicate, with ON-TARGETplus™ Non-Targeting siRNA (Thermo Scientific, Waltham, Mass.) as a negative control, with biological replicates. For experiments with downstream protein analyses, cells were transfected with siRNAs as above. 60 hours post-transfection, cells were treated with 300 nM, 1 μM, or 4 μM GDC-0941 for 24 hours prior to harvesting. Cell viability was measured 72 hours later using the CellTiter 96® AQueous One Solution Cell Proliferation Assay (Promega, Madison, Wis.). siRNA knockdown after 72 hours was validated by Western Blotting using primary antibodies to OGT (Sigma, St. Louis, Mo.) or qRT-PCR using the Roche LightCycler® 480 (Roche, Indianapolis, Ind.) as described previously (Harradine et al., Mol. Cancer Res. 9:173-182, 2011) using the following primers to DDN (5-TGAACAGTGGTAGCGACAGC-3′ (forward) (SEQ ID NO:1), 5′-GGAGCTATCTCGGTGCCTG-3′ (reverse) (SEQ ID NO:2), 5′-FAM-ATCCCCAAGCCAAAGCTACAGGGA-3′-BHQ (probe) (SEQ ID NO:3)) and to the endogenous control, ACTB (5′-CAGCAGATGTGGATCAGCAAG-3′ (forward) (SEQ ID NO:4), 5′-GCATTTGCGGTGGACGAT-3′ (reverse) (SEQ ID NO:5), 5′-FAM-AGGAGTATGACGAGTCCGGCCCC-3′-BHQ (probe) (SEQ ID NO:6)).

Phosphorylation, Apoptosis Assays, and Western Blotting

Phosphorylation and/or cleavage status were determined as described above.

For Western analysis, cells were washed with PBS, and lysate was collected using the M-PER® extraction reagent (Thermo Scientific, Waltham, Mass.) containing HALT™ Protease and Phosphatase Inhibitor Cocktail (Thermo Scientific, Waltham, Mass.). 20 μg of protein per sample were separated by electrophoresis on 4-12% Bis-Tris NuPAGE® Novex acrylamide gels (Invitrogen, Grand Island, N.Y.) and were then transferred to nitrocellulose membranes using the iBlot® system (Invitrogen, Grand Island, N.Y.). Antibodies used were OGT, O-GlcNAc, β-actin, GSK-3β (S9), p27Kip1, FoxM1, FoxO1 (T24)/FoxO3a (T32), and total Cyclin D1. OGT and O-GlcNAc antibodies were obtained from Sigma (St. Louis, Mo.). All other antibodies were obtained from Cell Signaling Technology (Danvers, Mass.). Quantitative analysis of pixel density for O-GlcNAc staining was determined using AlphaVIEW™ SA software (Cell Biosciences, Santa Clara, Calif.).

Loss of OGT Expression and Concomitant Decreased O-GlcNAc Levels Sensitizes Tumor Cell Lines to Phosphoinositide 3-Kinase Inhibitors and Alters Multiple Signaling Pathways

OGT is a glycosyltransferase that, in response to cellular glucose levels and receptor tyrosine kinase activation, reversibly modifies diverse cytosolic and nuclear proteins through the addition of O-linked β-N-acetylglucosamine (O-GlcNAc) (Slawson et al., Nat. Rev. Cancer 11:678-684, 2011; Wells et al., Science 291:2376-2378). The O-GlcNAc modification acts as a regulatory switch in a manner analogous to phosphorylation, and has been shown to influence protein-protein interactions, glucose metabolism, proteasome function, transcriptional machinery, and mechanisms of cellular invasion and proliferation in breast cancer (Zhang et al., Cell 115:715-725, 2003; Yang et al., Nature 451:964-969, 2008; Caldwell et al., Oncogene 29:2831-2842, 2010; Vosseller et al., Proc. Natl. Acad. Sci. USA 99:5313-5318, 2002). OGT was one of the top differentially expressed genes that distinguished between GDC-0941-sensitive and GDC-0941-resistant tumor cell lines (Tables 3 and 4; FIGS. 4A and 4B).

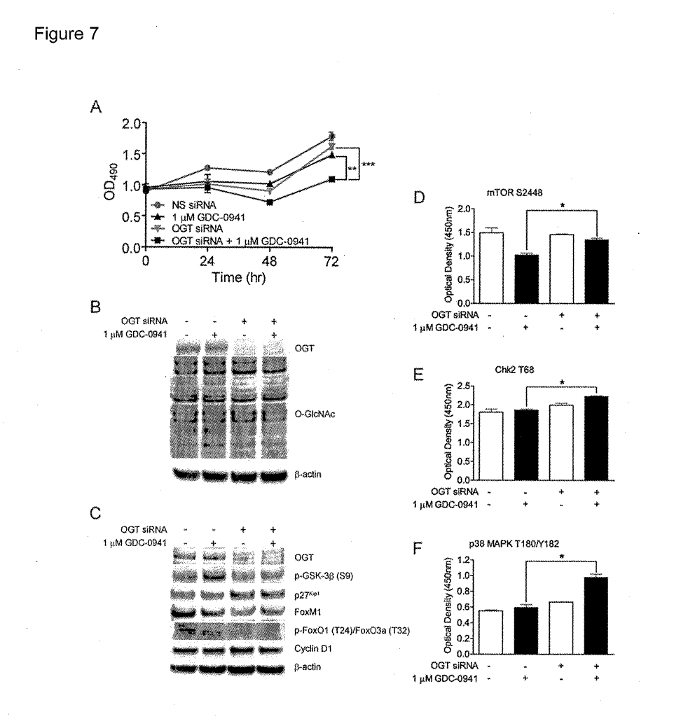

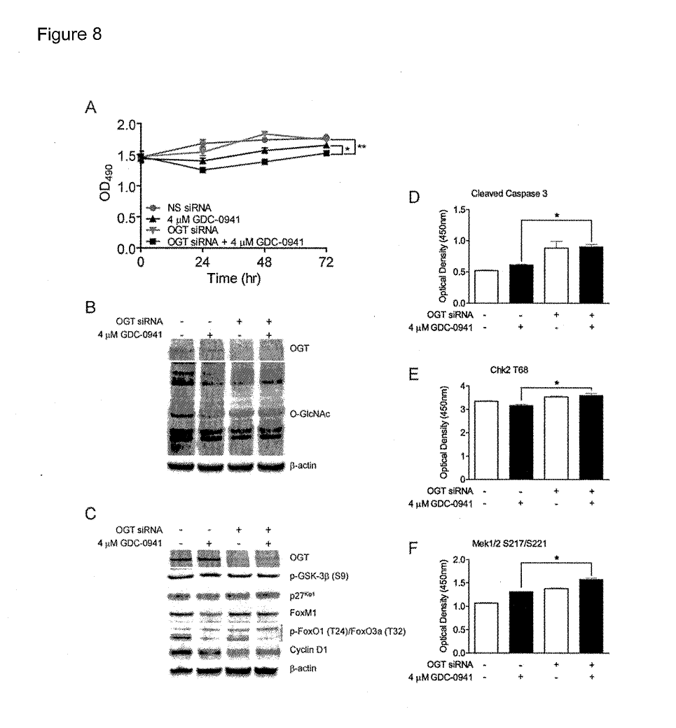

To assess the role of OGT in mediating sensitivity to a PI3K inhibitor, such as GDC-0941, siRNA pools targeting OGT were used to knockdown the expression of OGT in the GDC-0941-resistant MDA-MB-231 and OVCAR-4 tumor cell lines. The cell lines were then assayed for resulting alterations in cell proliferation and phosphorylation status of multiple signaling pathways, including downstream effectors of PI3K (FIGS. 7 and 8). Knockdown of OGT in both cell lines resulted in statistically significant enhanced sensitivity to GCD-0941 as compared to control cells only treated with either GDC-0941 or OGT siRNA (FIGS. 7A and 8A).

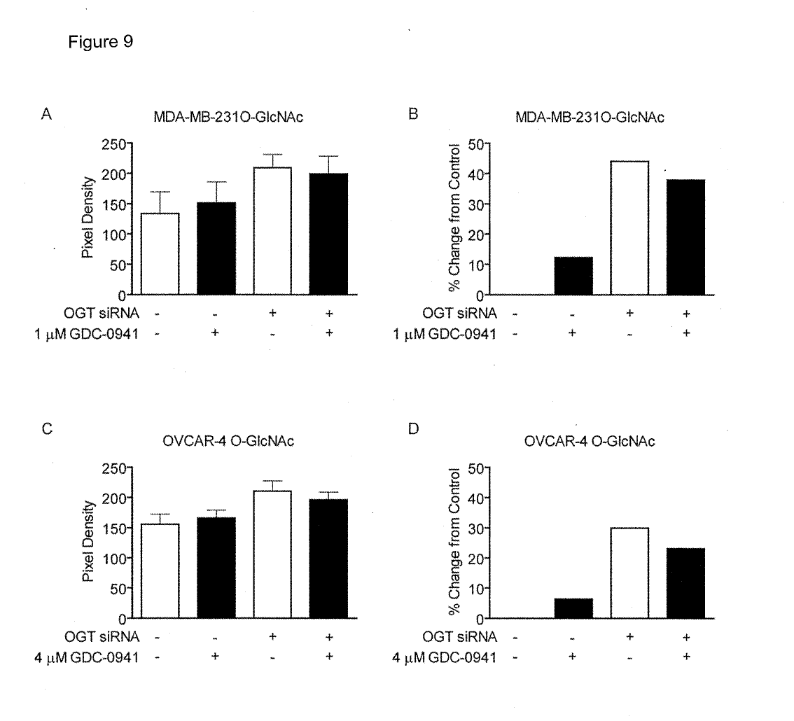

Next, alterations in cellular signaling pathways that may be driving the increased sensitivity observed upon loss of OGT expression in the MDA-MB-231 and OVCAR-4 tumor cell lines were probed. Immunoblotting of cell lysates from both MDA-MB-231 and OVCAR-4 cell lines treated with GDC-0941 and OGT siRNA, showed that upon the loss of OGT expression, there is concomitant decrease in total cellular levels of O-GlcNAc (FIGS. 7B and 8B; FIG. 9). In the MDA-MB-231 cell line treated with OGT-siRNAs and 1 μM GDC-0941, significant alterations were observed in multiple substrates of Akt1, including decreased phosphorylation of both GSK-3β and FoxO1/FoxO3a, as well as increased levels of total p27Kip1, relative to GDC-0941- or OGT siRNA-alone treated cells (FIG. 7C). In contrast, while similar decreased levels of phosphorylated FoxO1/FoxO3a were observed in OVCAR-4 cells treated with OGT-siRNAs and 4 μM GDC-0941, levels of phosphorylated GSK-3β and total p27Kip1 were unchanged (FIG. 8C). However, decreased levels of total Cyclin D1 were observed in OVCAR-4 tumor cells treated with OGT siRNA and 4 μM GDC-0941, relative to GDC-0941- or OGT siRNA-alone treated cells (FIG. 8C).

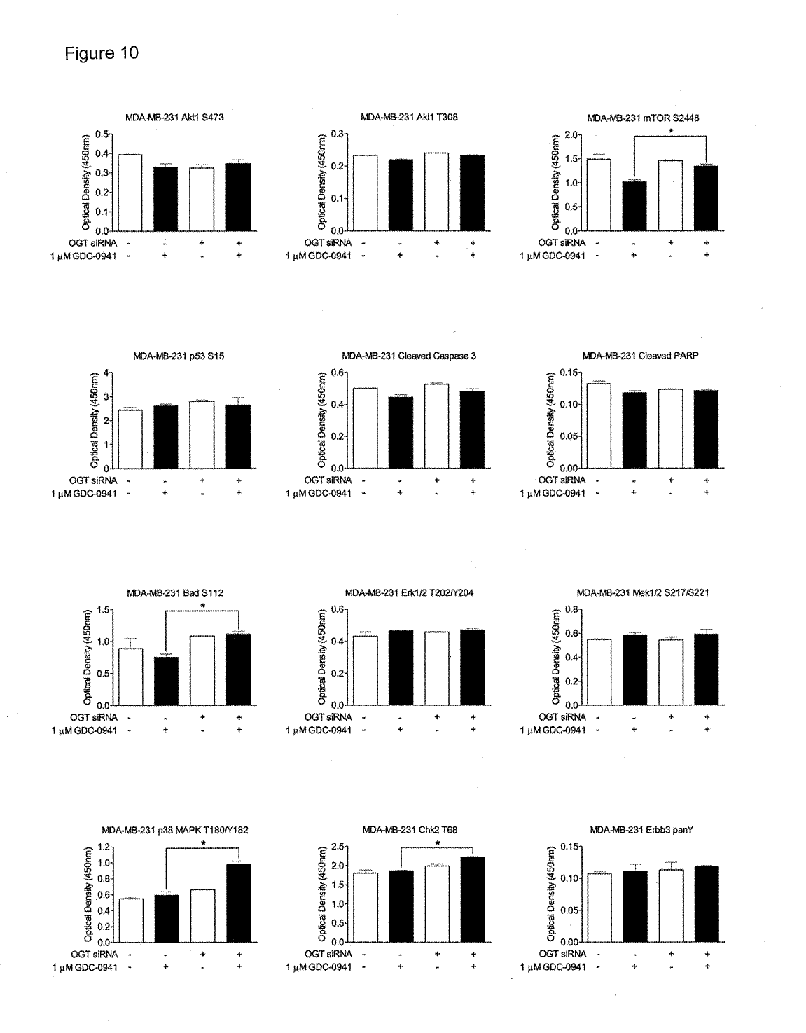

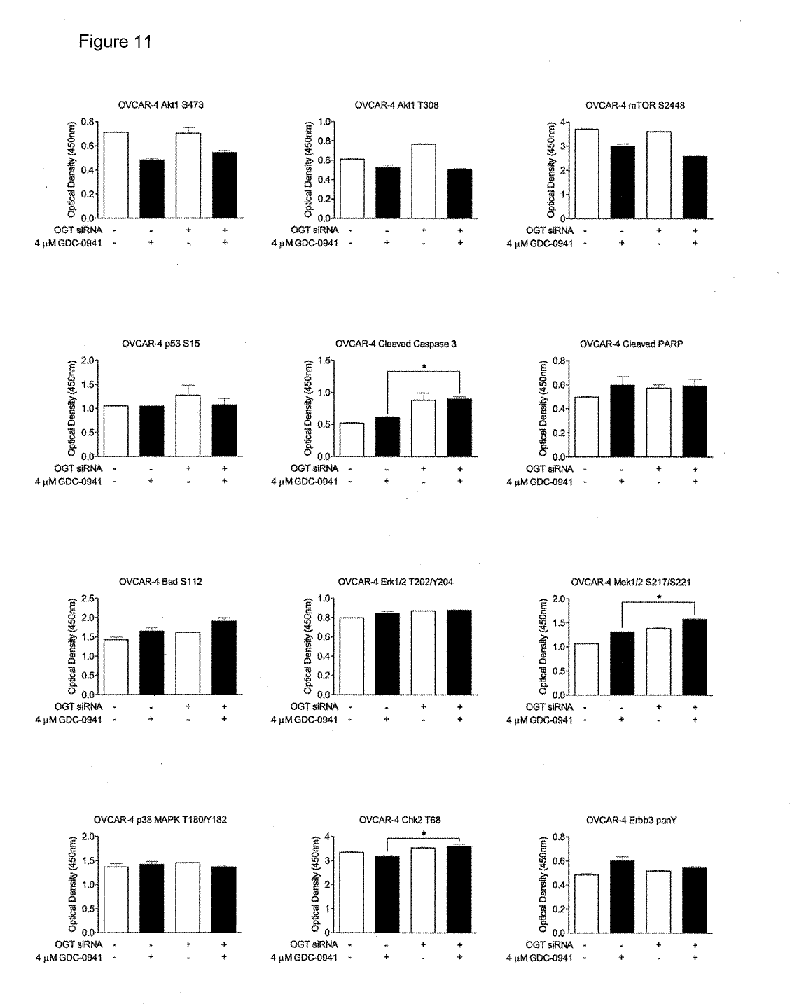

The effects of loss of OGT expression were further analyzed by examining the phosphorylation status of additional signaling nodes, including key regulators of apoptotic and MAPK pathways. While variable alterations were observed in pathway effectors in the MDA-MB-231 and OVCAR-4 cell lines upon treatment with GDC-0941 and OGT siRNAs (FIGS. 10 and 11), increased phosphorylation of the cell cycle checkpoint regulator Chk2 was observed in both cell lines (FIGS. 7E and 8E), relative to control cells. Interesting, in both the MDA-MB-231 and OVCAR-4 cell lines treated with OGT siRNA and GDC-0941, there was no observed alteration in phosphorylation of Akt1, either on Ser473 or Thr308 (FIGS. 10 and 11).

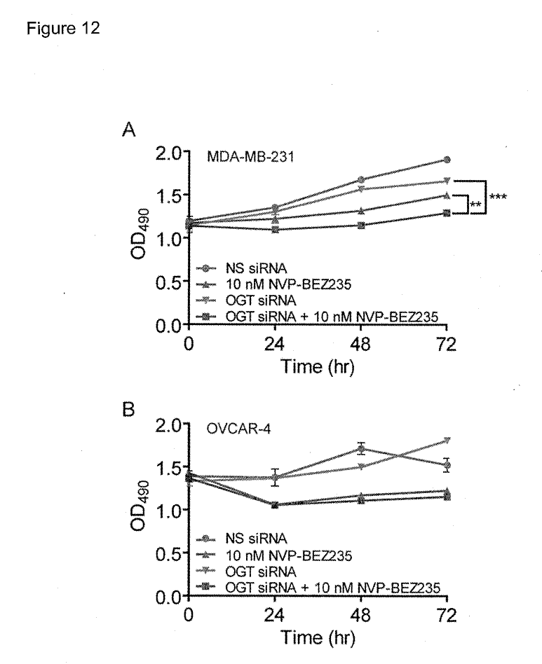

To further investigate the role of OGT in regulating sensitivity to inhibitors of the PI3K pathway, the effects that the loss of OGT expression had on tumor cell line sensitivity to the dual PI3K/mTOR inhibitor, NVP-BEZ235 (Maira et al., Mol. Cancer Ther. 7:1851-1863, 2008) were analyzed. In the NVP-BEZ235-resistant MDA-MB-231 breast tumor cell line (data not shown), loss of OGT expression resulted in increased sensitivity to NVP-BEZ235 (FIG. 12A). In contrast, loss of OGT expression had no effect in the sensitivity of the highly NVP-BEZ235-resistant OVCAR-4 ovarian tumor cell line (data not shown) to NVP-BEZ235 (FIG. 12B).

Loss of DDN Expression Sensitizes Breast Tumor Cell Lines to GDC-0941 and Alters Multiple Signaling Pathways

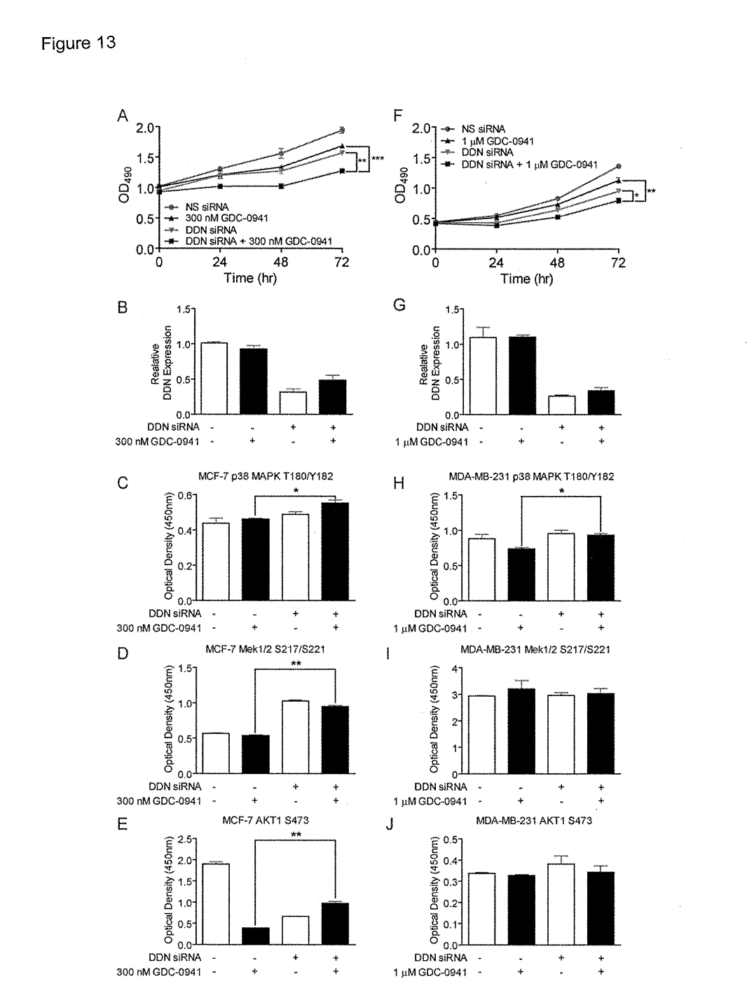

Dendrin (DDN) is a proline-rich protein that is thought to play a role in the TGF-β1-induced apoptosis of podocytes in the kidney (Asanuma et al., Proc. Natl. Acad. Sci. USA 104:10134-10139, 2007), possibly through its function as a transcription factor that promotes the cytosolic expression of cathepsin L (Yaddanapudi et al., J. Clin. Invest. 121:3965-3980, 2011). To assess the role of DDN in mediating sensitivity to inhibition of PI3K by GDC-0941, siRNA pools targeting DDN were used to knockdown the expression of DDN in the GDC-0941-sensitive MCF-7 breast tumor cell line and in the GDC-0941-resistant MDA-MB-231 breast tumor cell line. The cell lines were then assayed for cell proliferation and phosphorylation status of multiple signaling pathways, including downstream effectors of PI3K (FIG. 13). Knockdown of DDN in both cell lines (FIGS. 6B and 6G) resulted in statistically significant enhanced sensitivity to GCD-0941 as compared to control cells only treated with either GDC-0941 or DDN siRNA (FIGS. 13A and 13F).

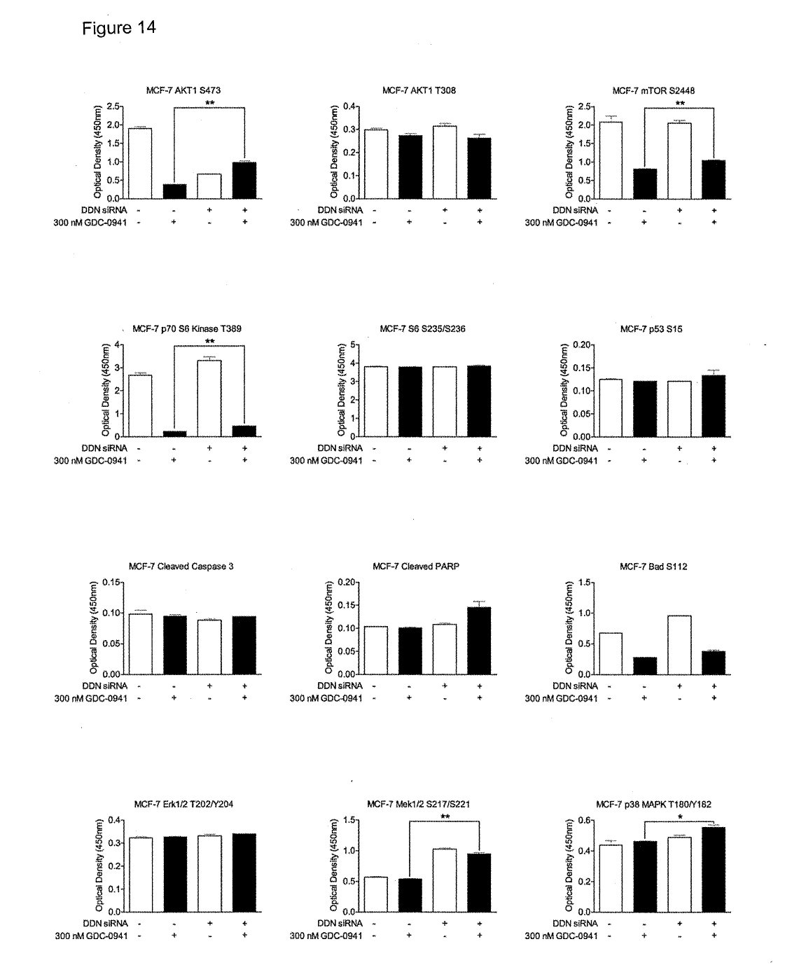

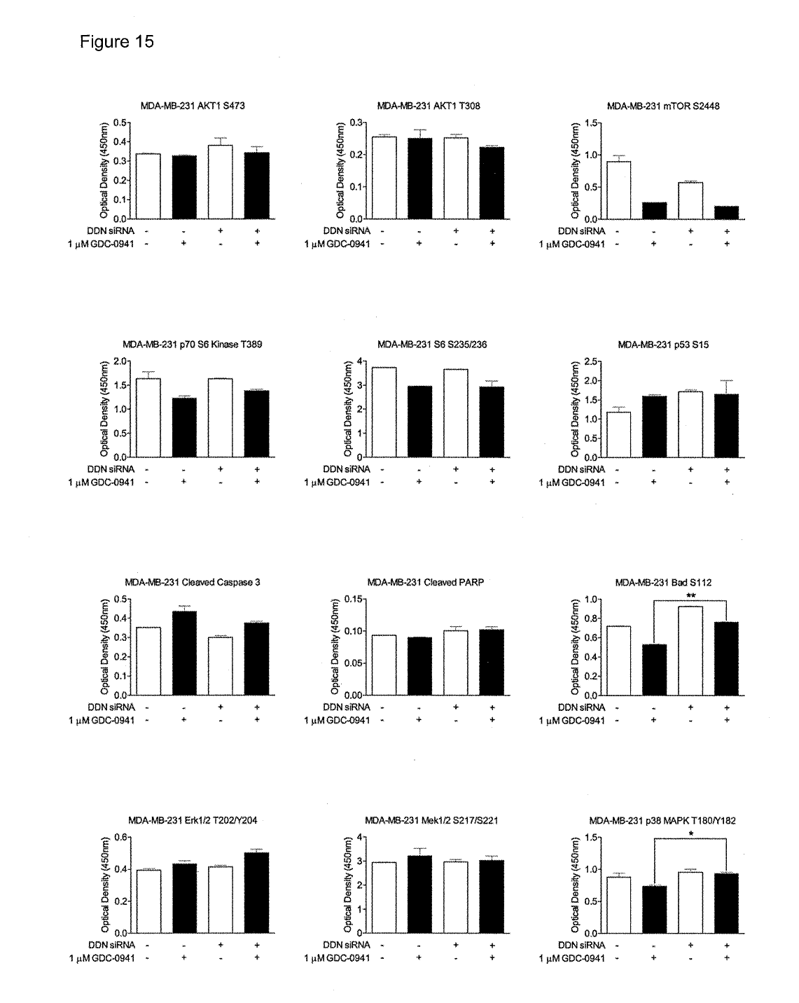

The effects of loss of DDN expression were analyzed by examining the phosphorylation status of additional signaling nodes, including key regulators of apoptotic and MAPK pathways. While variable alterations were observed in pathway effectors in the MCF-7 and MDA-MB-231 cell lines upon treatment with GDC-0941 and DDN siRNAs (FIGS. 14 and 15), increased phosphorylation of p38 MAPK at T180/Y182 was observed in both cell lines (FIGS. 13C and 13G), relative to control cells. Interesting, in the de novo GDC-0941-sensitive cell line, MCF-7, significant alterations were observed in the phosphorylation of Mek ½ at S217/S221 (FIG. 13C), Aktl at Ser473, and mTOR at 52448 (FIG. 14). No similar changes were observed in the de novo GDC-0941-resistant cell line, MDA-MB-231 (FIG. 13H; FIG. 15).

In conclusion, these data show that GDC-0941 has broad anti-proliferative activity in the nine tumor types represented in the NCI-60 cell line panel, suggesting its clinical utility against multiple tumor types, and that tumor cell lines with activated PI3K pathway signaling, independent of PI3K mutation status, are highly sensitive to inhibition of PI3K with GDC-0941. Numerous genomic and phsophoproteomic biomarkers have also been identified that are predictive of sensitivity to a PI3K inhibitor, such as GDC-0941. These data also highlight several biomarkers that may serve as pharmacodynamics markers of drug efficacy, or as additional drug targets to increase patient response to PI3K inhibitors.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method for predicting a likelihood that a tumor cell will be sensitive or resistant to a phosphoinositide 3-kinase (PI3K) inhibitor, comprising:
a. assaying an expression level of one or more genes selected from Table 2 or Table 3 in a tumor cell; and
b. predicting a likelihood that the tumor cell will be sensitive to the PI3K inhibitor, wherein increased expression level of the one or more genes selected from Table 2 compared to the expression level of the one or more genes selected from Table 2 in a PI3K inhibitor-resistant tumor cell is positively correlated with a likelihood that the tumor cell will be sensitive to the PI3K inhibitor, or predicting a likelihood that the tumor cell will be resistant to the PI3K inhibitor, wherein increased expression level of the one or more genes selected from Table 3 compared to the expression level of the one or more genes selected from Table 3 in a PI3K inhibitor-susceptible tumor cell is positively correlated with a likelihood that the tumor cell will be resistant to the PI3K inhibitor.

2. The method of claim 1, wherein the one or more genes selected from Table 2 is selected from VAV3, DGKE, MBD1, SERP1, TXNL1, NLGN4X, C1ORF91, SLC25A1, ZBTB40, FAM51A1, LOC116349, KIAA1468, PIGN, PKD1L1, SELT, CISH, MGC50559, NPY5R, PEX10, and C6ORF35.

3. The method of claim 1, further comprising increasing the sensitivity of the tumor cell to the PI3K inhibitor, comprising, contacting the tumor cell with an activator of the one or more genes selected from Table 2, and contacting the tumor cell with a PI3K inhibitor.

4. (canceled)

5. The method of claim [[4]] 1, wherein the one or more genes selected from Table 3 is selected from TRIM50C, GALR2, INSL3, LOC389633, GTPBP10, PGRMC1, DNASE1L3, CACNG2, FAM90A1, OGT, FKBP6, GDAP1L1, CHRNB1, NLGN3, ZNF259, DDN, NXF3, MGC35366, TANK, and LOC116123.

6. The method of claim 1, further comprising increasing the sensitivity of the tumor cell to the PI3K inhibitor, comprising, contacting the tumor cell with an inhibitor of the one or more genes selected from Table 3, and contacting the tumor cell with the PI3K inhibitor.

7. The method of claim 1, wherein the expression level of the one or more genes selected from Table 2 or Table 3 is normalized against an expression level of one or more reference genes to obtain a normalized expression level of the one or more genes selected from Table 2 or Table 3.

8. The method of claim 1, wherein the expression level of the one or more genes selected from Table 2 or Table 3 is a level of RNA transcript of the one or more genes selected from Table 2 or Table 3.

9. The method of, wherein the expression level of the one or more genes selected from Table 2 or Table 3 is a polypeptide level of the one or more genes selected from Table 2 or Table 3.

10. The method of claim 8, wherein the level of RNA transcript of the one or more genes selected from Table 2 or Table 3 is assayed using reverse transcription polymerase chain reaction (RT-PCR).

11. The method of claim 1, wherein the tumor cell is from a biopsy sample.

12. The method of claim 1, wherein the tumor cell is from a fixed, wax-embedded tissue sample.

13. The method of claim 1, further comprising creating a report based on the normalized expression level of the one or more genes selected from Table 2 or Table 3.

14. A method of increasing sensitivity of a tumor cell to a PI3K inhibitor, comprising,
contacting the tumor cell with an inhibitor of one or more genes selected from Table 3; and
contacting the tumor cell with a PI3K inhibitor.

15. A method of increasing sensitivity of a tumor cell to a PI3K inhibitor, comprising,
contacting the tumor cell with an activator of one or more genes selected from Table 2; and
contacting the tumor cell with a PI3K inhibitor.

16. The method of claim 1, wherein the tumor cell is selected from a breast, colon, NSCLC, renal, ovarian, prostate, and melanoma tumor cell.

17. The method of claim 1, wherein the PI3K inhibitor is GDC-0941.
